(19) United States
(12) Patent Application Publication
(10) Pub. No.: US 2009/0137493 A1
(43) Pub. Date: May 28, 2009

(54) Inhibition of tumor metastases using protein kinase C (PKC) inhibitors

(76) Inventors: Daria D. Mochly-Rosen, Menlo Park, CA (US); Jeewon Kim, Palo Alto, CA (US); Steve Thorne, Pittsburgh, PA (US)

Correspondence Address: King & Spalding LLP, P.O. Box 889, Belmont, CA, 94002-0889, US

(21) Appl. No.: 12/157,408

(22) Filed: Jun. 9, 2008

Related U.S. Application Data
(60) Provisional application No. 60/933,801, filed on Jun. 7, 2007.

(51) Int. Cl. A61K 38/08; A61P 35/04

(52) U.S. Cl. 514/16; 514/17

(57) ABSTRACT

Described are methods for reducing tumor metastasis in an animal by administering an inhibitor of a protein kinase C (PKC) isozyme.

### PRIORITY

The present application claims priority to U.S. Provisional Application Ser. No. 60/933,801, filed Jun. 7, 2007, which is hereby incorporated by reference in its entirety

### STATEMENT REGARDING GOVERNMENT INTEREST

This work was supported in part by National Institute of Health, grant number P50 CA114747. Accordingly the United States government may have certain rights in this invention.

### TECHNICAL FIELD

The subject matter described herein relates to methods for reducing tumor metastasis using an inhibitor of a protein kinase C (PKC) isozyme.

### BACKGROUND

Metastatic cancers spread (i.e., metastasize) from their original site to one or more remote sights in the body. Virtually all cancers have the potential to spread this way, although whether metastases develop depends on complex interactions involving many factors, including the type of cancer, the degree of maturity (differentiation) of the tumor cells, the location of the tumor, how long the cancer has been present, and other factors.

Tumor cells appear to metastasize through several mechanisms, for example, by local extension from the tumor to the surrounding tissues, via the bloodstream to distant sites, or via the lymphatic system to neighboring or distant lymph nodes. Different types of tumor may exhibit characteristic routes of metastasis, which may involve a combination of mechanisms.

The preferred treatment for a metastatic cancer largely depends on where the cancer started. For example, when breast cancer spreads to the lungs it remains a breast cancer and treatment is determined by the tumor's origin within the breast, not by the fact that the tumor cells are now present in the lung. However, in about five-percent of cases, metastases are discovered without identifying the primary tumor. In such cases, treatment is typically dictated by the metastatic location.

Although the presence of metastases generally implies a poor prognosis, some metastatic cancers can be cured with conventional therapy. Early detection and diagnosis improves the chances of successful treatment. Symptoms vary according to the type of cancer and the metastatic sites involved. Many patients have no or minimal symptoms related to the tumor and their metastases, which are found only during a routine medical evaluation.

Protein kinase C (PKC) is a key enzyme in signal transduction involved in a variety of cellular functions, including cell growth, regulation of gene expression, and ion channel activity. The PKC family of isozymes includes at least 11 different protein kinases that can be divided into at least three subfamilies based on their homology and sensitivity to activators. Each isozyme includes a number of homologous conserved (“C”) domains interspersed with isozyme-unique variable (“V”) domains. Members of the classical PKC (cPKC) subfamily, i.e., α, β_{I}, β_{II}, and γPKC, contain four homologous domains (C1, C2, C3 and C4) and require calcium, phosphatidylserine, and diacylglycerol or phorbol esters for activation. Members of the novel PKC (nPKC) subfamily, i.e., δ, ε, η, and θPKC, lack the C2 homologous domain and do not require calcium for activation. Finally, members of the atypical PKC (aPKC) subfamily, i.e., ζ and λ/iPKC, lack both the C2 homologous domain and one-half of the C1 homologous domain, and are insensitive to diacylglycerol, phorbol esters, and calcium.

Studies on the subcellular distribution of PKC isozymes demonstrate that activation of PKC results in its redistribution (also called translocation) within a cell, such that activated PKC isozymes associate with the plasma membrane, cytoskeletal elements, nuclei, and other subcellular compartments (Saito, N. et al., Proc. Natl. Acad. Sci. USA 86:3409-3413 (1989); Papadopoulos, V. and Hall, P. F. J. Cell Biol. 108:553-567 (1989); Mochly-Rosen, D., et al., Molec. Biol. Cell (formerly Cell Reg.) 1:693-706, (1990)), while inactive PKC isozymes tend to be found in the cytosol. The unique cellular functions of different PKC isozymes are determined by their subcellular location. For example, activated β_{I}PKC is found in the nucleus, whereas activated β_{II}PKC is found at the perinucleus and cell periphery of cardiac myocytes (Disatnik, M. H., et al., Exp. Cell Res. 210:287-297 (1994)). εPKC, whose activation requires phospholipids but is independent from calcium, is found in primary afferent neurons both in the dorsal root ganglia as well as in the superficial layers of the dorsal spinal cord.

The different cellular localization of PKC isozymes appears to be due to binding of the activated isozymes to specific anchoring molecules termed Receptors for Activated C-Kinase (“RACKs”). RACKs are thought to function by selectively anchoring activated PKC isozymes to their respective subcellular sites. RACKs bind only fully activated PKC and are not necessarily substrates of the enzyme. Nor is the binding to RACKs mediated via the catalytic domain of the kinase (Mochly-Rosen, D., et al., Proc. Natl. Acad. Sci. USA 88:3997-4000 (1991)). Translocation and binding to an appropriate RACK is required to produce its characteristic cellular responses (Mochly-Rosen, D., et al., Science 268:247-251 (1995)). Conversely, inhibition of PKC binding to RACK in vivo inhibits PKC translocation and PKC-mediated function (Johnson, J. A., et al., J. Biol. Chem., 271:24962-24966 (1996a); Ron, D., et al., Proc. Natl. Acad. Sci. USA 92:492-496 (1995); Smith, B. L. and Mochly-Rosen, D., Biochem. Biophys. Res. Commun., 188:1235-1240 (1992)).

Individual PKC isozymes have been implicated in the mechanisms of various disease states, including cancer (i.e., α and δ PKC); cardiac hypertrophy and heart failure (i.e., β_{I} and β_{II}PKC); nociception (i.e., γ and εPKC); ischemia, including myocardial infarction (i.e., δPKC); immune response, particularly T-cell mediated (i.e., θPKC); and fibroblast growth and memory (i.e., ζPKC). Various PKC isozyme- and variable region-specific peptides have been previously described (see, e.g., U.S. Pat. No. 5,783,405). The role of εPKC in pain perception has recently been reported (WO 00/01415; U.S. Pat. No. 6,376,467), including therapeutic use of the εV1-2 peptide (a selective inhibitor of εPKC first described in U.S. Pat. No. 5,783,405). The binding specificity for RACK1, a selective anchoring protein for β_{II}PKC, has recently been reported to reside in the V5 region of β_{II}PKC (Stebbins, E. et al., J. Biol. Chem. 271:29644-29650 (2001)), which study included testing certain N-terminus, middle, and C-terminus peptides alone, in combination, and together with a mixture of three peptides from the βC2 domain.

Notwithstanding such reported advances, new, selective agents and methods for the treatment of disease, including alternatives to known PKC isozyme- and variable region-specific peptides, continue to be desired.

The foregoing examples of the related art and limitations related therewith are intended to be illustrative and not exclusive. Other limitations of the related art will become apparent to those of skill in the art upon a reading of the specification and a study of the drawings.

### REFERENCES

Each of the following references, as well as other reference cited herein, are hereby incorporated by reference in their entirety:
Bogenrieder, T., and Herlyn, M. (2003). Axis of evil: molecular mechanisms of cancer metastasis. Oncogene 22, 6524-6536.
Brule, S., Charnaux, N., Sutton, A., Ledoux, D., Chaigneau, T., Saffar, L., and Gattegno, L. (2006). The shedding of syndecan-4 and syndecan-1 from HeLa cells and human primary macrophages is accelerated by SDF-1/CXCL12 and mediated by the matrix metalloproteinase-9. Glycobiology 16, 488-501.
Hauzenberger, D., Klominek, J., Holgersson, J., Bergstrom, S. E., and Sundqvist, K. G. (1997). Triggering of motile behavior in T lymphocytes via cross-linking of alpha 4 beta 1 and alpha L beta 2. J Immunol 158, 76-84.
Hehlgans, S., Haase, M., and Cordes, N. (2007). Signalling via integrins: implications for cell survival and anticancer strategies. Biochim Biophys Acta 1775, 163-180.
Ivaska, J., Kermorgant, S., Whelan, R., Parsons, M., Ng, T., and Parker, P. J. (2003). Integrin-protein kinase C relationships. Biochem Soc Trans 31, 90-93.
Larsson, C. (2006). Protein kinase C and the regulation of the actin cytoskeleton. Cell Signal 18, 276-284.
Mostafavi-Pour, Z., Askari, J. A., Parkinson, S. J., Parker, P. J., Ng, T. T., and Humphries, M. J. (2003). Integrin-specific signaling pathways controlling focal adhesion formation and cell migration. J Cell Biol 161, 155-167.
Ng, T., Shima, D., Squire, A., Bastiaens, P. I., Gschmeissner, S., Humphries, M. J., and Parker, P. J. (1999). PKCalpha regulates beta 1 integrin-dependent cell motility through association and control of integrin traffic. Embo J 18, 3909-3923.
Parsons, M., Keppler, M. D., Kline, A., Messent, A., Humphries, M. J., Gilchrist, R., Hart, I. R., Quittau-Prevostel, C., Hughes, W. E., Parker, P. J., and Ng, T. (2002). Site-directed perturbation of protein kinase C-integrin interaction blocks carcinoma cell chemotaxis. Mol Cell Biol 22, 5897-5911.
Rigot, V., Lehmann, M., Andre, F., Daemi, N., Marvaldi, J., and Luis, J. (1998). Integrin ligation and PKC activation are required for migration of colon carcinoma cells. J Cell Sci 111 (Pt 20), 3119-3127.
Smith, M. C., Luker, K. E., Garbow, J. R., Prior, J. L., Jackson, E., Piwnica-Worms, D., and Luker, G. D. (2004). CXCR4 regulates growth of both primary and metastatic breast cancer. Cancer Res 64, 8604-8612.
Thodeti, C. K., Albrechtsen, R., Grauslund, M., Asmar, M., Larsson, C., Takada, Y., Mercurio, A. M., Couchman, J. R., and Wewer, U. M. (2003). ADAM12/syndecan-4 signaling promotes beta 1 integrin-dependent cell spreading through protein kinase Calpha and RhoA. J Biol Chem 278, 9576-9584.
Ways, D. K., Kukoly, C. A., deVente, J., Hooker, J. L., Bryant, W. O., Posekany, K. J., Fletcher, D. J., Cook, P. P., and Parker, P. J. (1995). MCF-7 breast cancer cells transfected with protein kinase C-alpha exhibit altered expression of other protein kinase C isoforms and display a more aggressive neoplastic phenotype. J Clin Invest 95, 1906-1915.

### BRIEF SUMMARY

The following aspects and embodiments thereof described and illustrated below are meant to be exemplary and illustrative, not limiting in scope.

In one aspect, a method for inhibiting metastasis is provided, comprising administering to a patient with a tumor an effective amount of a protein kinase C (PKC) inhibitor.

In some embodiments, the PKC inhibitor is an αPKC inhibitor. In some embodiments, the αPKC inhibitor comprises an amino acid sequence from the αPKC V5 domain. In particular embodiments, the αPKC inhibitor comprises the αV5-3 peptide (SEQ ID NO: 6). In some embodiments, the αV5-3 peptide is conjugated to a peptide for increase cell permeability.

In some embodiments, the PKC inhibitor is a β_{II}PKC inhibitor. In some embodiments, the β_{II}PKC inhibitor comprises an amino acid sequence from the β_{II}PKC V5 domain. In particular embodiments, the β_{II}PKC inhibitor comprises the β_{II}v5-3 peptide (SEQ ID NO: 13). In some embodiments, the β_{II}v5-3 peptide is conjugated to a peptide for increase cell permeability.

In some embodiments, the PKC inhibitor is an εPKC inhibitor. In some embodiments, the εPKC inhibitor comprises an amino acid sequence from amino acid residues 14-21 of εPKC (SEQ ID NO: 20). In particular embodiments, the εv1-2 peptide is conjugated to a peptide for increase cell permeability.

In some embodiments, the tumor is a breast cancer tumor. In some embodiments, the tumor is a mammary tumor.

In addition to the exemplary aspects and embodiments described above, further aspects and embodiments will become apparent by reference to the drawings and by study of the following descriptions.

### BRIEF DESCRIPTION OF THE DRAWINGS

FIG. 1A shows an autoradiogram from an immunoblot assay of the cytosol and particulate fractions of 4T1 (metastatic) and JC (non-metastatic) mouse mammary cancer cells probed with anti-αPKC antibodies.

FIG. 1B is a bar graph showing the percentage of translocation of the αPKC isozyme from the cytosol to the particulate cell fraction, based on the immunoblot analysis of FIG. 1A, for the 4T1 and JC cells.

FIG. 2A shows an autoradiogram from an immunoblot assay of the cytosol and particulate fractions of 4T1 tumors grown in mice and fractionated, and probed with anti-αPKC antibodies, anti-β_{II}PKC antibodies, anti-δPKC antibodies, or anti-εPKC antibodies.

FIG. 2B is a bar graph showing the percentage of translocation of the αPKC, β_{II}PKC, δPKC, and εPKC isozymes from the cytosol to the particulate cell fraction in the 4T1 tumor fractionates, based on the immunoblot analysis of FIG. 2A.

FIG. 3 shows the results of imaging a representative mouse from each group of tumor-bearing mice treated for four weeks with saline, TAT peptide (TAT), and αV5-3-TAT conjugate peptide (αPKC).

FIG. 4 is a bar graph showing the extent of lung metastasis, expressed as relative light units (based on imaging as exemplified in FIG. 3), in tumor-bearing mice treated for four weeks with saline, the αV5-3-TAT conjugate peptide (αPKC), or the β_{II}V5-3-TAT conjugate peptide (βPKC).

FIGS. 5A-5B are bar graphs showing the percentage of translocation of αPKC (FIG. 5A) and β_{II}PKC (FIG. 5B) from the particulate fraction to the cytosol in tumor cells treated with αV5-3-TAT peptide.

FIG. 6A is a bar graph showing the affect of saline, the αV5-3-TAT conjugate peptide (αPKC), or the β_{II}V5-3-TAT conjugate peptide (βPKC) on the growth rate of primary tumors in vivo.

FIGS. 6B-6C are bar graphs showing the affect of the αV5-3-TAT peptide on growth rate of JC tumor cells (FIG. 6B) and 4T1 tumor cells (FIG. 6C) in vitro. The cells treated with the TAT carrier peptide (TAT) or with αV5-3-TAT peptide (PKC alpha inhibitor), at doses of 0, 1, 5, and 10 μM.

FIG. 7A is a bar graph showing the affect of αV5-3-TAT peptide on the adhesion of tumor cells into the lungs. Animals were treated with TAT carrier peptide (TAT), or αV5-3-TAT peptide (PKC alpha inhibitor), initiated two days before, and continued for 12 days following, injection of tumor cells intravenously;

FIGS. 7B-7C are images of mice two days after treatment as described in FIG. 7A. The mice in FIG. 7B were treated with TAT and the mice in FIG. 7C treated with αV5-3-TAT peptide. The imaging shows adhesion of tumor cells that migrated from the site of injection.

FIGS. 7D-7E are images of mice following five days of treatment as described in FIG. 7A. The mice in FIG. 7D were treated TAT (control) and the mice in FIG. 7E were treated with αV5-3-TAT peptide (PKC a inhibitor). Imaging shows adhesion of tumor cells that have migrated from the injection site to the lung.

FIGS. 8A-8B are computer-generated photomicrographs of lung tissue from mice four weeks following fatpad implantation of tumor cells. Osmotic pumps were implanted 1 week after fatpad implantation, for delivering TAT (control; FIG. 8A) or αV5-3-TAT peptide (PKC a inhibitor; FIG. 8B).

FIGS. 8C-8D are computer-generated photomicrographs of lung tissue from mice two weeks after intravenous injection of tumor cells. Osmotic pumps were implanted two days prior to fatpad implantation, for delivering TAT (control; FIG. 8C) or αV5-3-TAT peptide (PKC a inhibitor; FIG. 8D).

FIG. 9 is a graph showing the percent surviving animals in the time (in days) following intravenous administration of tumor cells. The mice were treated with saline (squares; PBS) or the αV5-3-TAT peptide (circles; peptide).

FIG. 10A is a bar graph showing the relative expression levels of beta 1 (β_{I}) integrin on the surface of tumor cells from animals with metastasis (Mets) or no metastasis (No mets).

FIG. 10B is a bar graph showing the relative expression levels of CXCR4 chemokine receptor on the surface of tumor cells from animals treated with the TAT carrier peptide (TAT) or the αV5-3-TAT peptide (Alpha inhibitor).

FIG. 10C is a bar graph showing the relative levels of matrix metalloproteinase 2 (MMP2) activity in tumor cells from animals treated with saline or TAT (untreated) or the αV5-3-TAT peptide (aV5-3).

FIGS. 11A-11B are bar graphs showing the relative serum levels of liver enzymes aspartate transaminase (AST; FIG. 11A) and alanine transaminase (ALT; FIG. 11B) in animals treated with saline or TAT (untreated) or the αV5-3-TAT peptide (aV5-3). FIGS. 11C-11E are bar graphs showing the relative serum levels of white blood cells (FIG. 11C), lymphocytes (FIG. 11D), and neutrophils (FIG. 11E).

FIGS. 12A-12D are micrographs showing the efficacy of TAT carrier peptide (TAT; FIG. 12A), αV5-3-TAT peptide (Alpha; FIG. 12B), β_{II}V5-3 peptide (Beta2; FIG. 12C), and εV1-2 (Epsilon; FIG. 12D) on human MDA-MB-231 breast cancer cell migration.

FIGS. 13A-13D are micrographs showing the efficacy of TAT carrier peptide (TAT; FIG. 13A), αV5-3-TAT peptide (Alpha; FIG. 13B), β_{II}V5-3 peptide (Beta2; FIG. 13C), and εV1-2 (epsilon; FIG. 13D) on human MCF-7 breast cancer cell migration.

FIGS. 14A and 14B are bar graphs showing the efficacy of TAT carrier peptide (TAT), αV5-3-TAT peptide (Alpha), β_{II}V5-3 peptide (Beta-2), and εV1-2 (Epsilon) in inhibiting the invasion of human MDA-MB-231 and MCF-7 breast cancer cells through a HUVEC monolayer. FIGS. 14C and 14D are bar graphs showing the efficacy of TAT carrier peptide (TAT) or αV5-3-TAT peptide in inhibiting the invasion of 4T1 (FIG. 11C) and MDA-MB-231 (FIG. 11D) cells.

FIGS. 15A-15D are micrographs showing the efficacy of TAT carrier peptide (TAT; FIG. 15A), αV5-3-TAT peptide (Alpha; FIG. 15B), β_{II}V5-3 peptide (Beta2; FIG. 15C), and εV1-2 (Epsilon; FIG. 15D) on JC cell migration.

FIGS. 16A-16D are micrographs showing the efficacy of TAT carrier peptide (TAT; FIG. 16A), αV5-3-TAT peptide (Alpha; FIG. 16B), β_{II}V5-3 peptide (Beta2; FIG. 16C), and εV1-2 (Epsilon; FIG. 16D) on 4T1 cell migration.

### BRIEF DESCRIPTION OF THE SEQUENCES

SEQ ID NO:1 represents the fifth variable (V5) domain of the human alpha protein kinase C (αPKC) isozyme:

PKVCGKGAENFDKFFTRGQPVLTPPDQLVIANIDQSDFEGFSYVNPQFVH
PILQSAV.

SEQ ID NO: 2 represents the fifth variable (V5) domain of the human beta-II protein kinase C (β_{II}PKC) isozyme:

PKACGRNAENFDRFFTRHPPVLTPPDQEVIRNIDQSEFEGFSFVNSEFL
KPEVKS

SEQ ID NO: 3 represents εPKC from Mus musculus; gi:6755084; ACCESSION: NP_{—}035234 XP_{—}994572 XP_{—}994601 XP_{—}994628.

SEQ ID NO: 4 represents εPKC from Rattus norvegicus; ACCESSION: NP_{—}058867 XP_{—}343013.

SEQ ID NO: 5 represents εPKC from Homo sapiens; ACCESSION: NP_{—}005391

SEQ ID NO: 6 is a peptide derived from SEQ ID NO:1, referred to herein as αV5-3, QLVIAN.

SEQ ID NO: 7 is a peptide derived from SEQ ID NO: 1, i.e., GKGAEN.

SEQ ID NO: 8 (i.e., QiVIAN); SEQ ID NO: 9 (i.e., QvVIAN); SEQ ID NO: 10 (i.e., QLVIAa); SEQ ID NO: 11 (QLVInN); and SEQ ID NO: 12 (QLVIAN) are derived from SEQ ID NOs: 1 and 6.

SEQ ID NO: 13 (CGRNAE); SEQ ID NO: 14 (KACGRNAE); SEQ ID NO: 15 (CGRNAEN); and modified peptide SEQ ID NO:16 (ACGkNAE) are β PKC inhibitors derived from SEQ ID NO: 2.

SEQ ID NO: 17 (ACGRNAE); SEQ ID NO: 18 (QEVIRN); and SEQ ID NO: 19 (SFVNSEFLKPEVKS) are also derived from SEQ ID NO: 2.

SEQ ID NO: 20 is a peptide from the first variable domain of epsilon PKC, more specifically from residues 14-21 of human εPKC, referred to as εV1-2; EAVSLKPT.

SEQ ID NO: 21 (HDAPIGYD), named ψεRACK, is a sequence in εPKC with 75% homology with a sequence in εRACK consisting of amino acids NNVALGYD (RACK 285-292; SEQ ID NO; 22).

SEQ ID NO: 23 (HNAPIGYD) is a mutated ψεRACK peptide that functions as an εPKC antagonist/inhibitor

SEQ ID NO: 24 (NNVALGYD) is an εPKC binding motif in the polypeptide β′-COP.

SEQ ID NO: 25 is a carrier peptide sequence from the Transactivating Regulatory Protein (TAT, amino acids 47-57 of TAT) from the Human Immunodeficiency Virus, Type 1, YGRKKRRQRRR

SEQ ID NO: 26 corresponds to the peptide inhibitor β_{II}V5-3 (SEQ ID NO: 6) attached via an N-terminal disulfide bond to TAT peptide, YGRKKRRQRRR-CC-QLVIAN.

SEQ ID NO: 27 corresponds to the peptide inhibitor β_{II}V5-3 (SEQ ID NO: 13) attached via an N-terminal disulfide bond to TAT peptide, YGRKKRRQRRR-CC-CGRNAE.

SEQ ID NO: 28 corresponds to the peptide inhibitor εV1-2 (SEQ ID NO: 20) attached via an N-terminal disulfide bond to TAT peptide, YGRKKRRQRRR-CC-EAVSLKPT.

SEQ ID NO: 29 is the Drosophila Antennapedia homeodomain-derived carrier peptide, RQIKIWFQNRRMKWKK.

### DETAILED DESCRIPTION

### I. Definitions

As used herein a “conserved set” of amino acids refers to a contiguous sequence of amino acids that is identical or closely homologous (e.g., having only conservative amino acid substitutions) between members of a group of proteins. A conserved set may be anywhere from two to over 50 amino acid residues in length. Typically, a conserved set is between two and ten contiguous residues in length.

As used herein, a “conservative amino acid substitutions” are substitutions that do not result in a significant change in the activity or tertiary structure of a selected polypeptide or protein. Such substitutions typically involve replacing a selected amino acid residue with a different residue having similar physico-chemical properties. For example, substitution of Glu for Asp is considered a conservative substitution since both are similarly-sized negatively-charged amino acids. Groupings of amino acids by physico-chemical properties are known to those of skill in the art.

As used herein, the terms “domain” and “region” are used interchangeably herein and refer to a contiguous sequence of amino acids within a PKC isozyme, typically characterized by being either conserved or variable.

As used herein, the terms “peptide” and “polypeptide” are used interchangeably herein and refer to a compound made up of a chain of amino acid residues linked by peptide bonds. Unless otherwise indicated, the sequence for peptides is given in the order from the “N” (or amino) termiums to the “C” (or carboxyl) terminus.

Two amino acid sequences or two nucleotide sequences are considered “homologous” (as this term is preferably used in this specification) if they have an alignment score of >5 (in standard deviation units) using the program ALIGN with the mutation gap matrix and a gap penalty of 6 or greater (Dayhoff, M. O., in Atlas of Protein Sequence and Structure (1972) Vol. 5, National Biomedical Research Foundation, pp. 101-110, and Supplement 2 to this volume, pp. 1-10.) The two sequences (or parts thereof) are more preferably homologous if their amino acids are greater than or equal to 50%, more preferably 70%, still more preferably 80%, identical when optimally aligned using the ALIGN program mentioned above.

A peptide or peptide fragment is “derived from” a parent peptide or polypeptide if it has an amino acid sequence that is homologous to the amino acid sequence of, or is a conserved fragment from, the parent peptide or polypeptide.

A “conserved set” of amino acids refers to a contiguous sequence of amino acids that is identical or closely homologous (e.g., having only conservative amino acid substitutions) between members of a group of proteins. A conserved set may be anywhere from two to over 50 amino acid residues in length. Typically, a conserved set is between two and ten contiguous residues in length. For example, for the two peptides CGRNAE (SEQ ID NO:15) and ACGRNAE (SEQ ID NO:19), there are 6 identical positions (CGRNAE) that form the conserved set of amino acids for these two sequences.

“Conservative amino acid substitutions” are substitutions that do not result in a significant change in the activity or tertiary structure of a selected polypeptide or protein. Such substitutions typically involve replacing a selected amino acid residue with a different residue having similar physico-chemical properties. For example, substitution of Glu for Asp is considered a conservative substitution since both are similarly-sized negatively-charged amino acids. Groupings of amino acids by physico-chemical properties are known to those of skill in the art.

“Domain” and “region” are used interchangeably herein and refer to a contiguous sequence of amino acids within a PKC isozyme, typically characterized by being either conserved or variable.

“Peptide” and “polypeptide” are used interchangeably herein and refer to a compound made up of a chain of amino acid residues linked by peptide bonds. Unless otherwise indicated, the sequence for peptides is given in the order from the “N” (or amino) terminus to the “C” (or carboxyl) terminus.

Two amino acid sequences or two nucleotide sequences are considered “homologous” (as this term is preferably used in this specification) if they have an alignment score of >5 (in standard deviation units) using the program ALIGN with the mutation gap matrix and a gap penalty of 6 or greater (Dayhoff, M. O., in ATLAS OF PROTEIN SEQUENCE AND STRUCTURE (1972) Vol. 5, National Biomedical Research Foundation, pp. 101-110, and Supplement 2 to this volume, pp. 1-10.) The two sequences (or parts thereof) are more preferably homologous if their amino acids are greater than or equal to 50%, more preferably 70%, still more preferably 80%, identical when optimally aligned using the ALIGN program mentioned above.

The term “effective amount” means a dosage sufficient to provide treatment for the disorder or disease state being treated. This will vary depending on the patient, the disease and the treatment being effected.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Abbreviations for amino acid residues are the standard 3-letter and/or 1-letter codes used in the art to refer to one of the 20 common L-amino acids.

As used herein and in the appended claims, the singular forms “a”, “an”, and “the” include plural reference, unless the context clearly dictates otherwise.

Unless defined otherwise, all technical and scientific terms used herein have the same meanings as commonly understood by one of ordinary skill in the art to which this subject matter belongs. Although any methods and materials similar or equivalent to those described herein can be used in the practice or testing of the present subject matter, the preferred methods, devices, and materials are now described.

All publications mentioned herein are incorporated herein by reference for the purpose of describing and disclosing the methodologies which are reported in the publications which might be used in connection with the subject matter herein.

Protein sequences are presented herein using the one letter or three letter amino acid symbols as commonly used in the art and in accordance with the recommendations of the IUPAC-IUB Biochemical Nomenclature Commission.

### II. Treatment Methods

The present methods provide PKC inhibitors for the inhibition of metastasis in an animal with a tumor. Inhibiting tumor metastasis includes reducing migration of tumor cells from the site of a primary tumor to a remote site; reducing signaling between tumor cells and remote sites in the body; and/or reducing the adhesion of tumor cells to remote sites in the body.

Studies and observations in support of the present methods are described below.

### A. Increased αPKC Activity of in Metastatic Mammary Cancer Cells

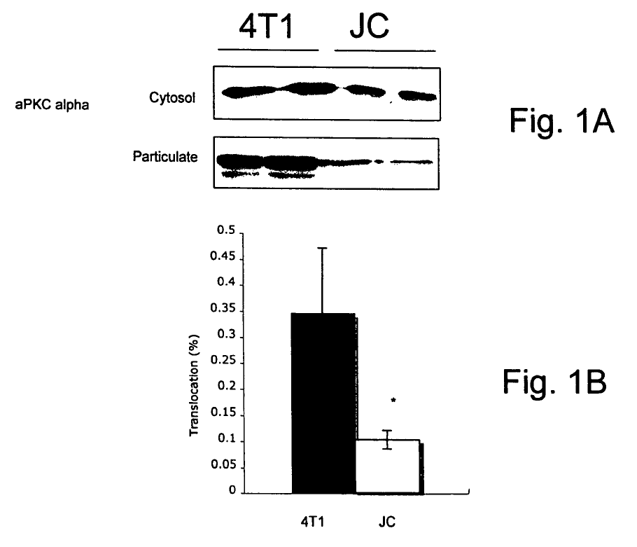

The present methods originated from the observation that the activity of αPKC in metastatic mammary cancer cells was significantly higher than in a similar non-metastatic cell line. The immunoblot of FIG. 1A and the graph of FIG. 1B, show the relative levels of αPKC in the cytosol and particulate fractions of 4T1 and JC mammary cancer cells, determined using anti-αPKC antibodies. The levels of activated (i.e., particulate) αPKC are several times higher in 4T1 metastatic mammary cancer cells than in JC non-metastatic mammary cancer cells.

### B. Higher Levels of Translocated/Activated αPKC and β_{II}PKC in Tumor Cells

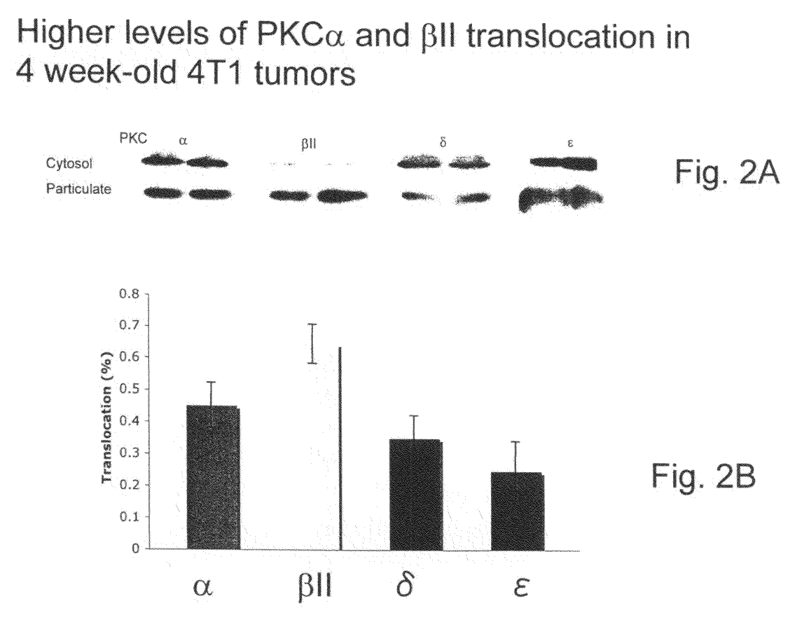

FIG. 2A show the results of a study in which 4T1 tumors were grown in mice, isolated, and then subjected to immunoblot analysis using antibodies specific for α, β_{II}, δ, or εPKC. FIG. 2B is a bar graph showing the percentage of translocation of the αPKC, β_{II}PKC, δPKC, and εPKC isozymes from the cytosol to the particulate cell fraction in the 4T1 tumor fractionates, based on the immunoblot analysis of FIG. 2A. The levels of translocation for the different PKC isozymes were β_{II}>α>δ>ε.

### C. αPKC and β_{II}PKC Inhibitors Reduces Tumor Metastasis in Animals

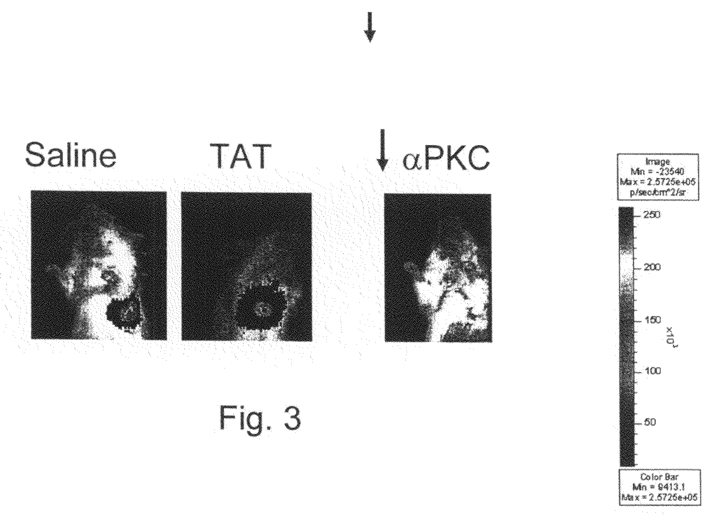

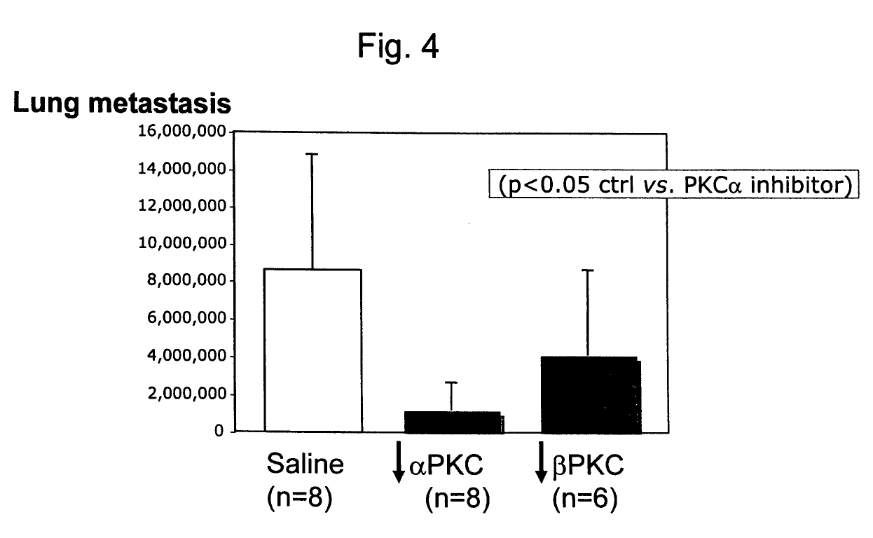

To determine whether specific PKC inhibitors could reduce the amount of PKC translocation/activation in vivo and thereby reduce tumor cell metastasis, animals were administered an αPKC peptide inhibitor, or appropriate control, via an implanted osmotic pump, following the injection of luciferase-tagged tumor cells. FIG. 3 shows the results of imaging a representative animal from each group of tumor-bearing mice treated for four weeks with saline, TAT peptide (TAT; SEQ ID NO: 25), and αV5-3-TAT conjugate peptide (αPKC; SEQ ID NO: 26). The data are summarized in the bar graph shown in FIG. 4, along with similar data relating to the use of a β_{II}V5-3-TAT conjugate peptide (βPKC; SEQ ID NO: 27). The extent of lung metastasis is expressed as relative light units. Treatment of mice with the αPKC inhibitor substantially reduced (i.e., about 4 to 5-fold) lung metastasis compared to that in control animals. Treatment of mice with the βPKC inhibitor reduced lung metastasis by about half, compared to that in control animals.

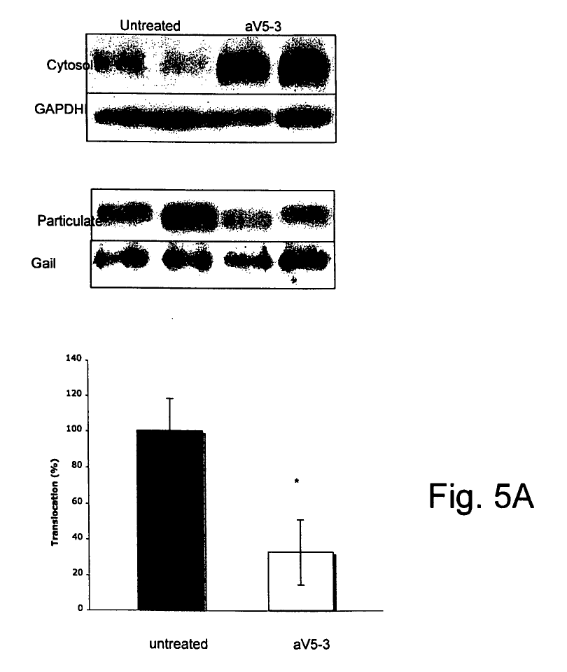

To further characterize inhibition of metastasis using the α and β_{II}PKC inhibitors, the percentage of translocation of αPKC (FIG. 5A) and β_{II}PKC (FIG. 5B), from the particulate fraction of the tumor cells to the cytosol was measured. As shown in FIG. 5A, treatment with a αPKC inhibitor reduced αPKC translocation by about 40%. However, treatment with a β_{II}PKC inhibitor (FIG. 5B) did not significantly reduce the translocation of β_{II}PKC, despite providing a measurable decrease in lung metastasis (as shown in FIG. 4).

### D. PKC α and βII Inhibitors do not Substantially Reduce Primarily Tumor Growth

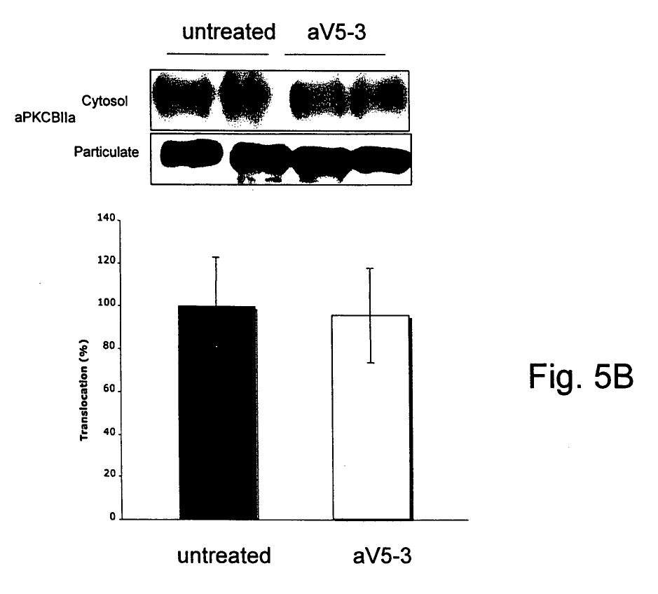

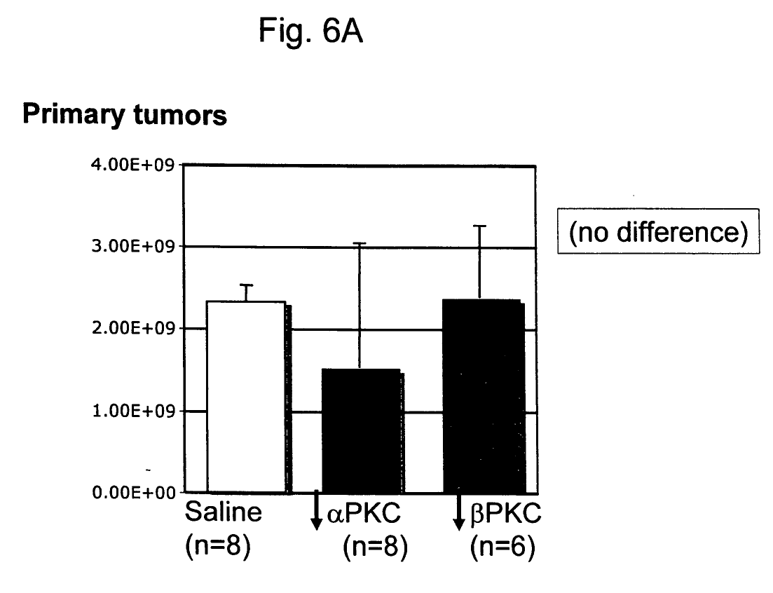

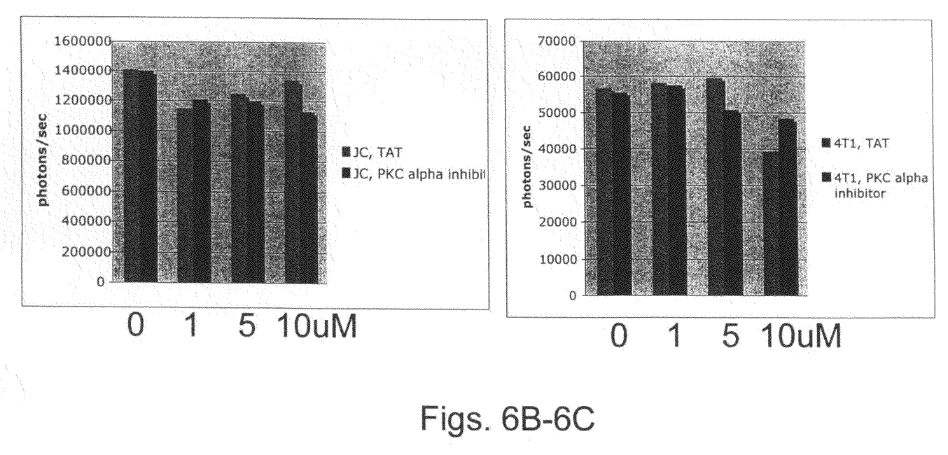

To determine if the effects observed with the α and β_{II}PKC peptide inhibitors was due to a decrease in metastasis as opposed to a decrease in primary tumor growth, animals injected with 4T1 tumor cells were treated with saline, the αV5-3-TAT conjugate peptide (αPKC), or the β_{II}V5-3-TAT conjugate peptide (βPKC) and the number of tumors cells at the primary site of injection were determined. As shown in FIG. 6A, no reduction in the number of cells at the primary tumor site was observed in animals treated with the β_{II}PKC inhibitor. Less than a two-fold reduction was observed in animals treated with the αPKC inhibitor, which was surprising in view of the significant effects of the αPKC inhibitor on metastasis. Results shown in FIGS. 6B-6C further show that the αV5-3-TAT peptide (PKC alpha inhibitor) does not substantially inhibit the growth of JC tumor cells (FIG. 6B) or 4T1 tumor cells (FIG. 6C) in vitro, compared to the TAT carrier peptide control (TAT).

### E. PKC Inhibitors Reduce Tumor Cell Migration/Invasion and Increase Survival Time

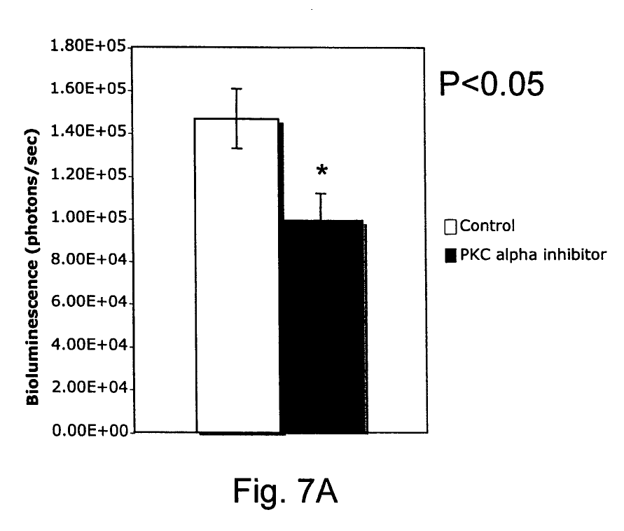

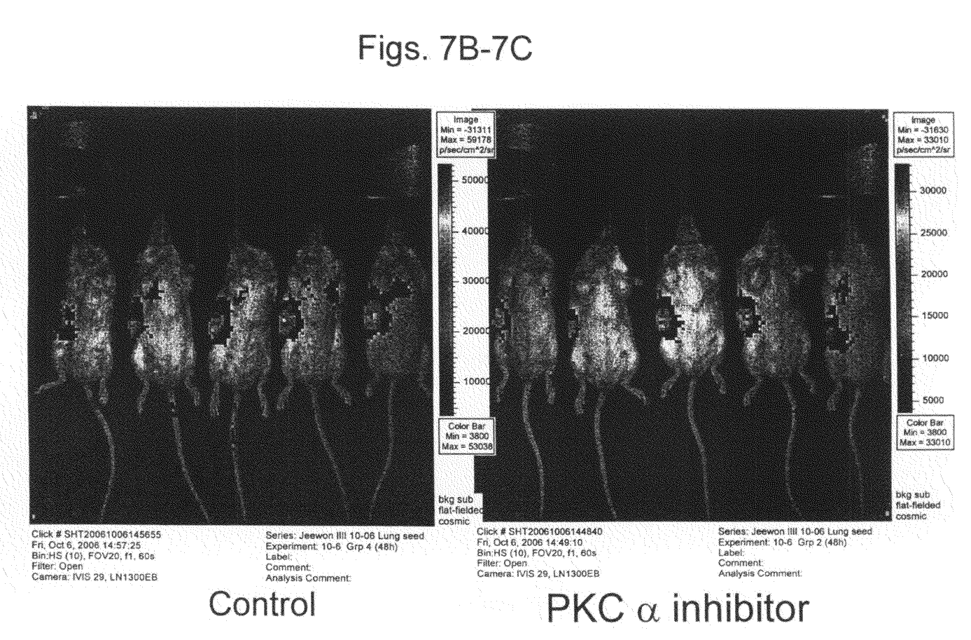

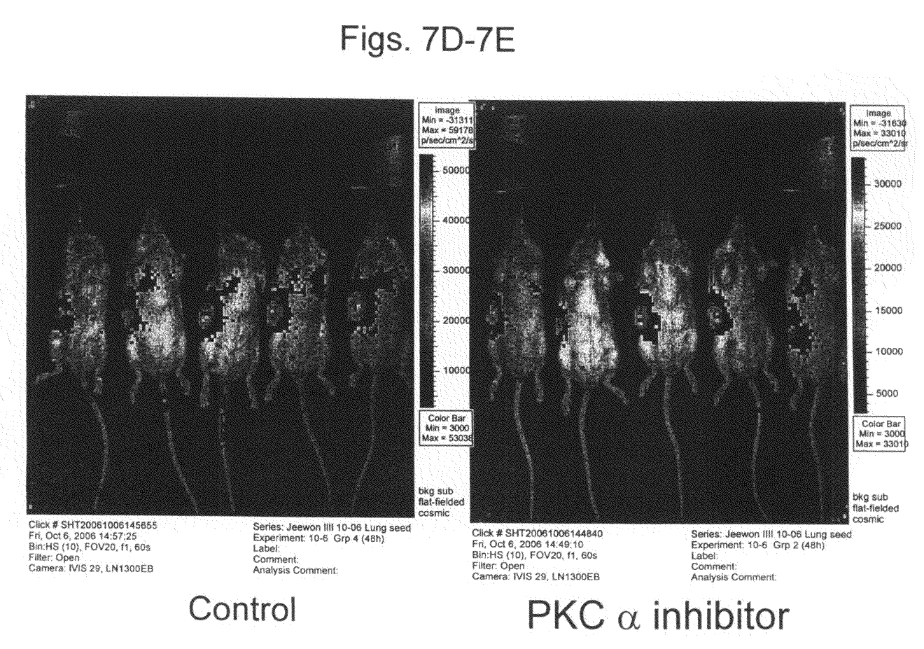

The results shown in FIGS. 5A-5B and 6B-6C indicated that the effect of PKC inhibitors in reducing metastasis was not primarily due to reducing the growth rate of the primary tumor cells. However, results of additional experiments elucidated the mechanism by which the PKC inhibitors reduced metastasis. For example, FIG. 7A is a bar graph showing the affect of αV5-3-TAT peptide on the adhesion of tumor cells into the lungs. In this experiment, animals were treated with TAT carrier peptide (TAT), or αV5-3-TAT peptide (PKC alpha inhibitor), for two days before injection of tumor cells intravenously. Adhesion of tumor cells to the lung was reduced in animals treated with the PKC alpha inhibitor compared to control animals.

FIGS. 7B and 7C are images of representative mice two days after treatment. The mice in FIG. 7B were treated with TAT and the mice in FIG. 7C treated with αV5-3-TAT peptide. The imaging shows reduced adhesion of tumor cells in the lung following treatment with the αV5-3-TAT peptide. FIGS. 7D-7E are images of mice following five days of treatment. The mice in FIG. 7D were treated TAT (control) and the mice in FIG. 7E were treated with αV5-3-TAT peptide (PKC a inhibitor). The imaging also shows reduced adhesion of tumor cells in the lung following treatment with the αV5-3-TAT peptide.

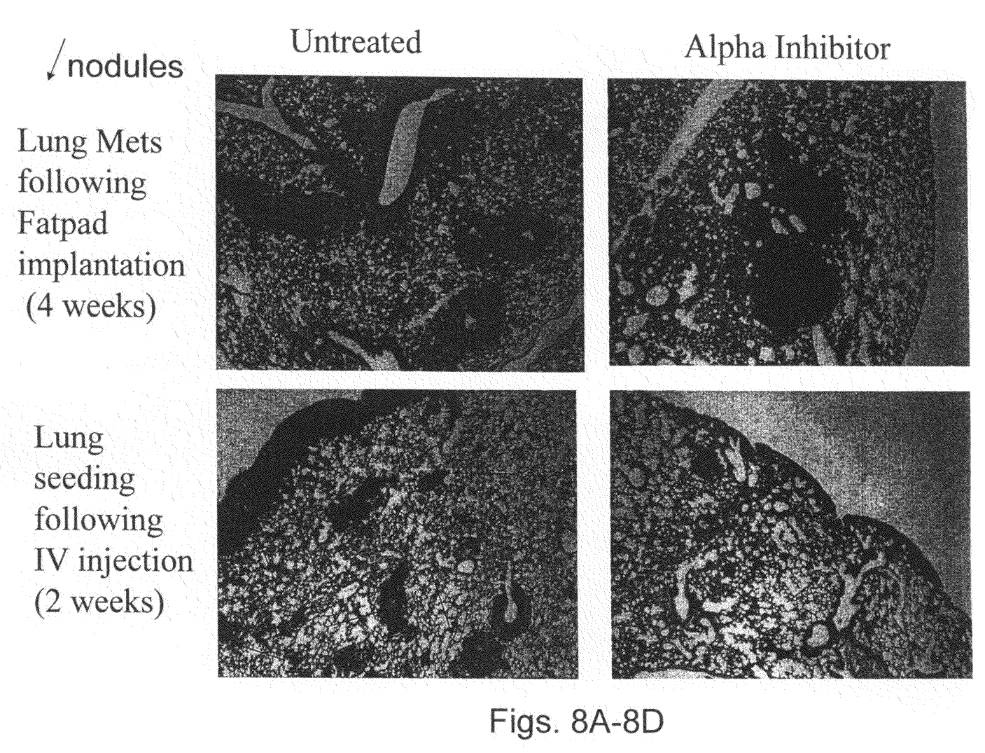

FIGS. 8A-8D are computer-generated photomicrographs of lung tissue from mice four weeks following fatpad implantation of tumor cells (FIGS. 8A-8B) or two weeks after intravenous injection of tumor cells (FIGS. 8C-8D). The histology results confirm those obtained using the luciferase imaging assay, i.e., that the PKC inhibitor reduced the invasion of lung tissues by tumor cells.

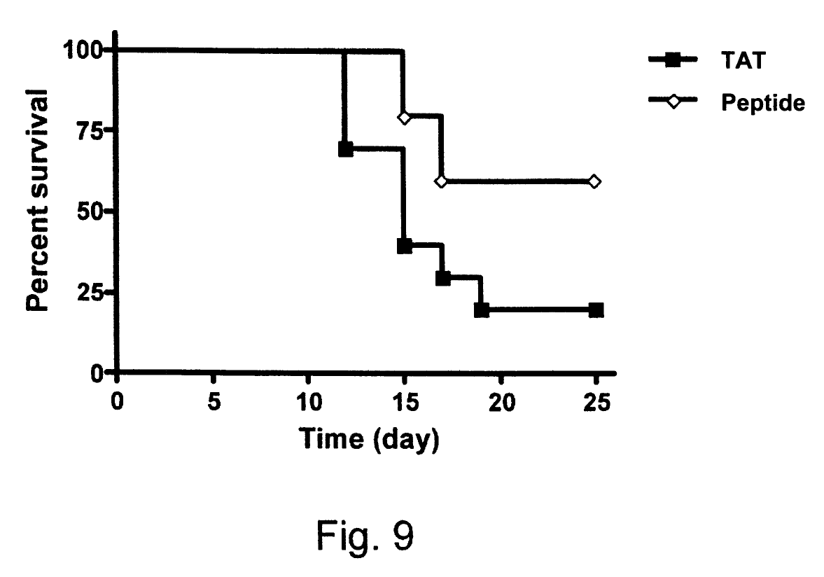

FIG. 9 is a graph showing the percent surviving animals in the time (in days) following intravenous administration of tumor cells. The mice were treated with saline (squares; PBS) or the αV5-3-TAT peptide (circles; peptide). The mice treated with the PKC inhibitor demonstrated longer survival times.

### F. Effect of Metastasis and PKC Inhibitors on the Expression and Activity of Other Cell Proteins

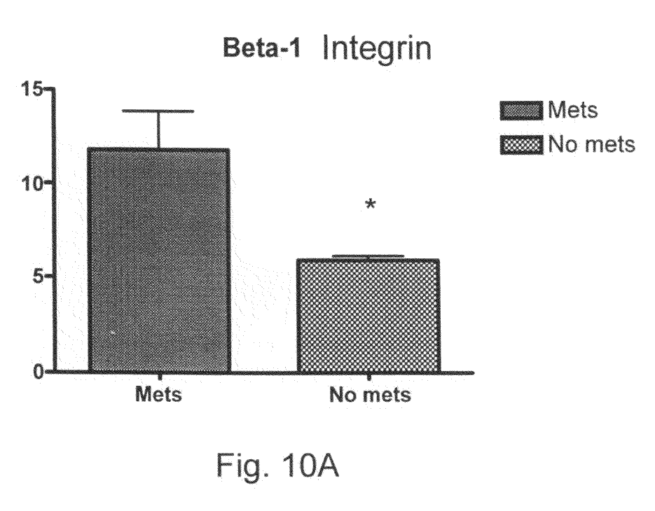

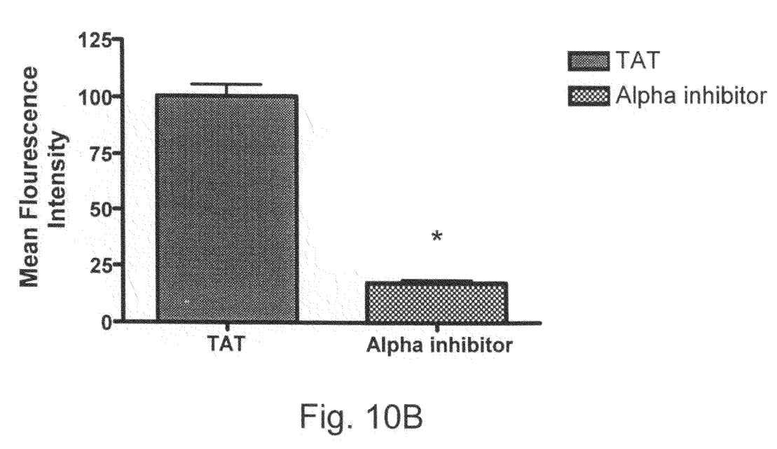

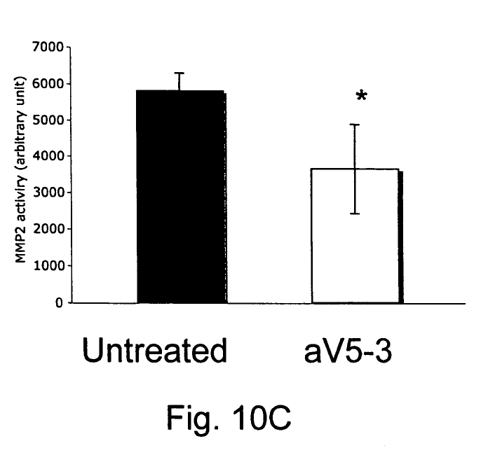

To better understand the role of PKC inhibitors in reducing metastasis, tumor cells, cells from primary tumors in animals with metastasis (Mets) and without metastasis (No mets) were isolated and assayed for levels of beta-1 integrin (FIG. 10A). The levels of beta-1 integrin were reduced about 50% in animals with no metastasis. The αV5-3-TAT peptide tended to decrease beta-1 integrin expression but the amount was not statistically significant (not shown).

Cells from primary tumors in animals treated with the αPKC inhibitor were also isolated and assayed for levels of CXCR4 (FIG. 10B) and levels of matrix metalloproteinase 2 (MMP2) activity (FIG. 10C). Treatment with the αV5-3-TAT substantially decreased (4-5-fold) the relative expression levels of CXCR4 chemokine receptor on the surface of tumor cells (FIG. 10B). Treatment with the αV5-3-TAT also decreased (about 30%) the relative levels of matrix metalloproteinase 2 (MMP2) activity in tumor cells (FIG. 10C).

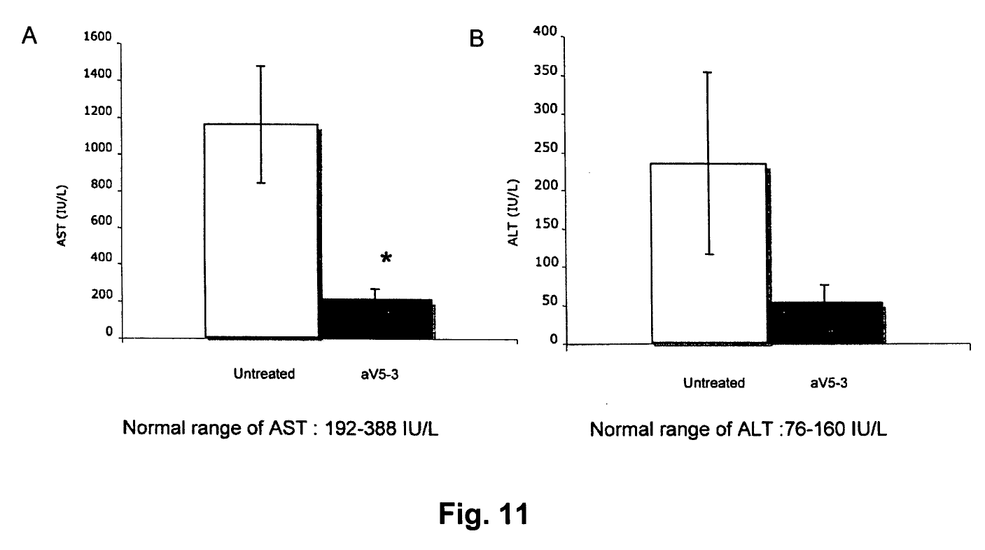

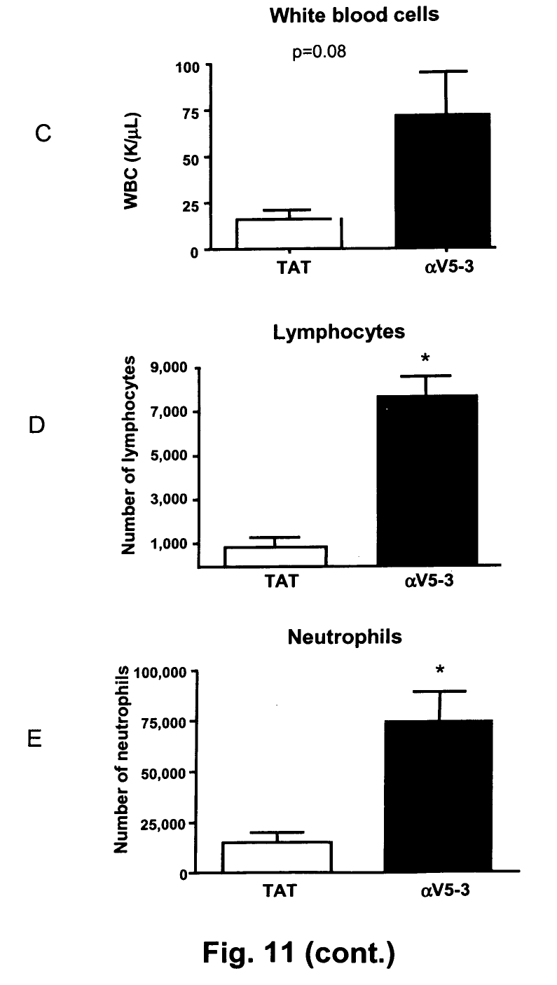

As shown in FIGS. 11A and 11B, treatment with the αV5-3-TAT also substantially decreased the relative serum levels of liver enzymes aspartate transaminase (AST; FIG. 11A; about 5-fold) and alanine transaminase (ALT; FIG. 11B; about 4-5-fold).

As further shown in FIGS. 11C-11E, no signs of immunosuppression were observed following treatment with the PKC inhibitor. Rather, the numbers of white blood cells (FIG. 11C), lymphocytes (FIG. 11D), and neutrophils (FIG. 11E) were all increased in αV5-3 treated animals compared to control (TAT) treated animals. These data suggest that PKC inhibition may have a direct immune-inducing activity, or may overcome tumor-mediated immune-suppression by reducing tumor burden.

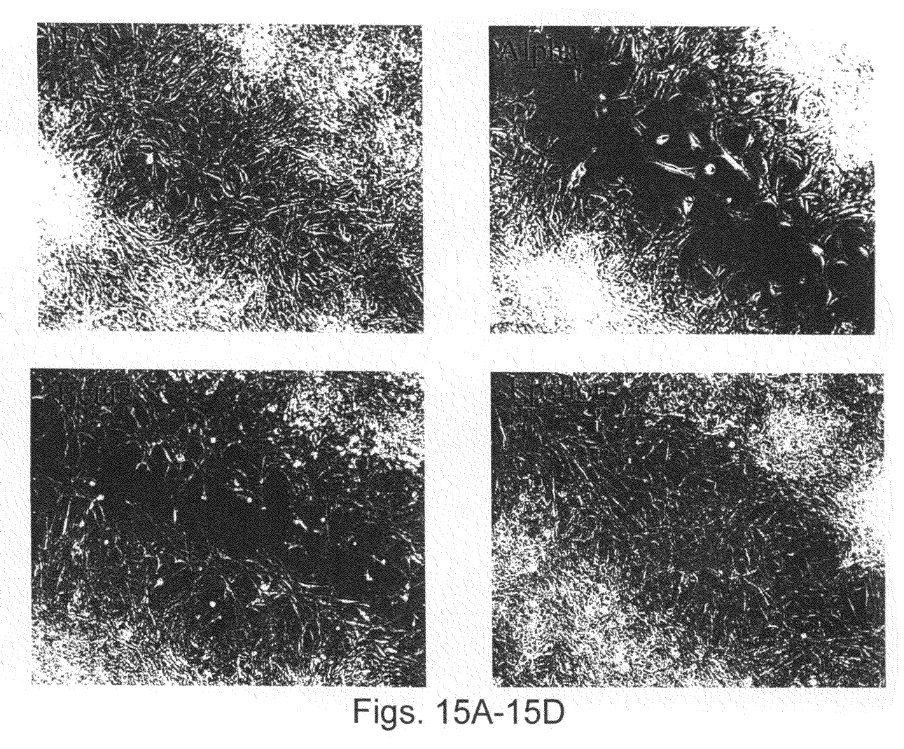

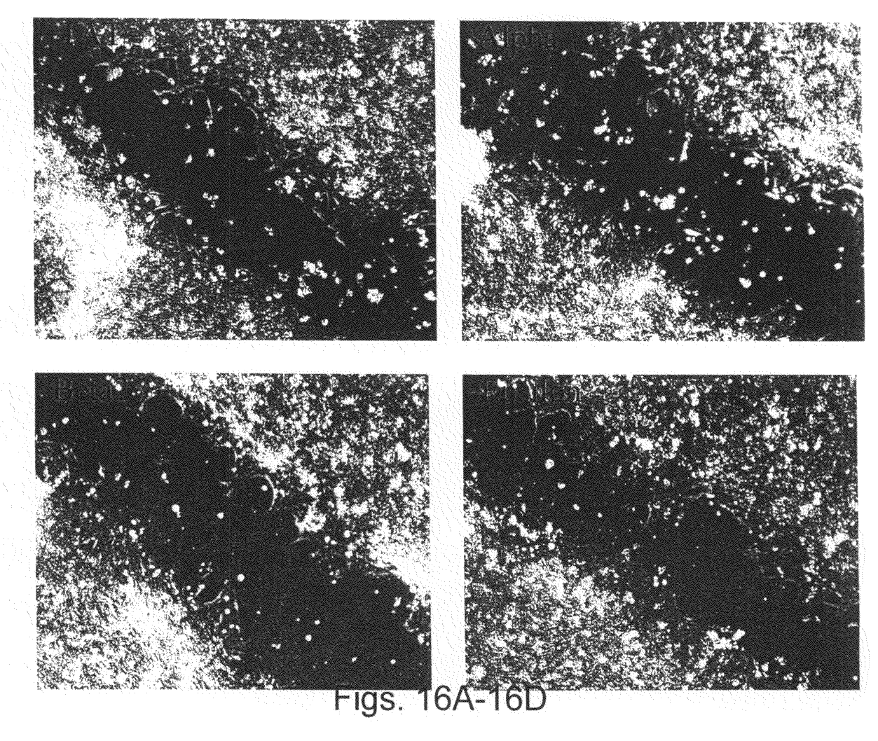

To investigate the role of PKC inhibitors in reducing the migration of tumor cells, in vitro migration assays were performed using MDA-MB-231 breast cancer cells (FIGS. 12A-12D), MCF-7 breast cancer cell (FIGS. 13A-13D), JC cells (FIGS. 15A-15D), and 4T1 cells (FIGS. 16A-16D), in the presence of TAT carrier peptide (TAT), αV5-3-TAT peptide (Alpha), β_{II}V5-3 peptide (Beta2), or εV1-2 peptide (Epsilon).

Treatment with the β_{II}V5-3 peptide (Beta2) reduced the migration of MDA-MB-231 breast cancer cells, MCF-7 breast cancer cells, and JC cells (FIGS. 12C, 13C, and 15C, respectively). Treatment with αV5-3-TAT peptide (Alpha) reduced the migration of JC cells (FIG. 15B).

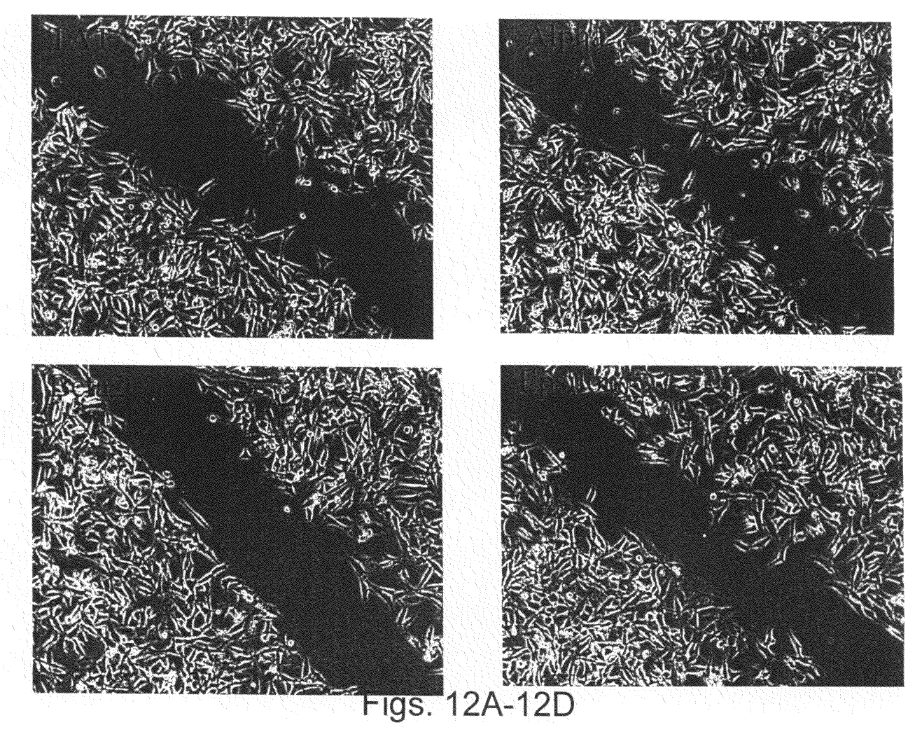

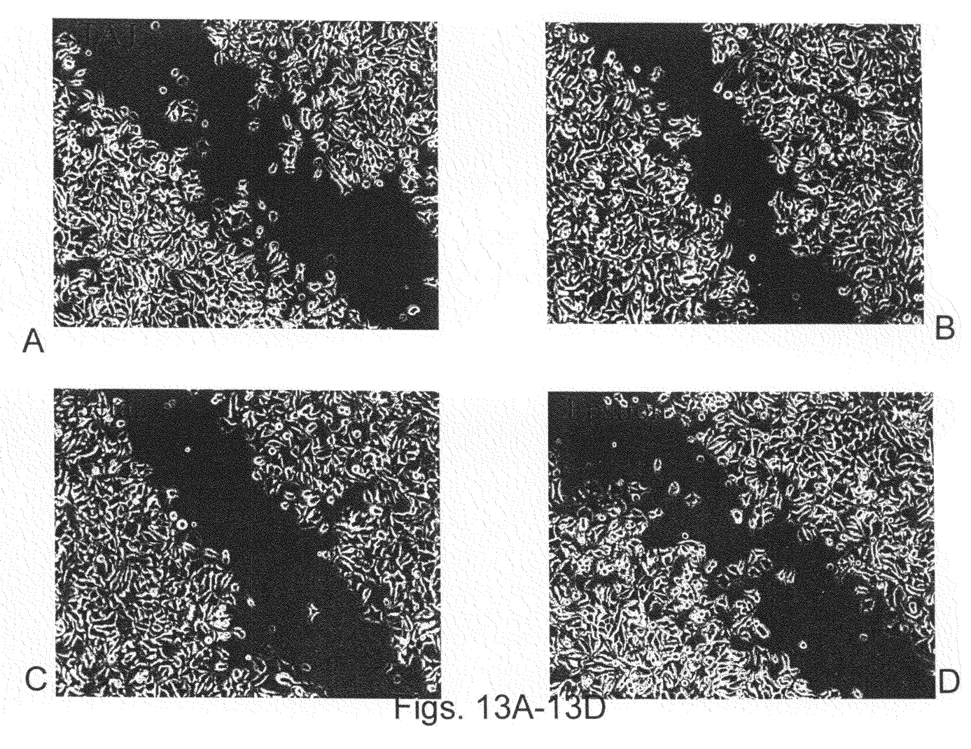

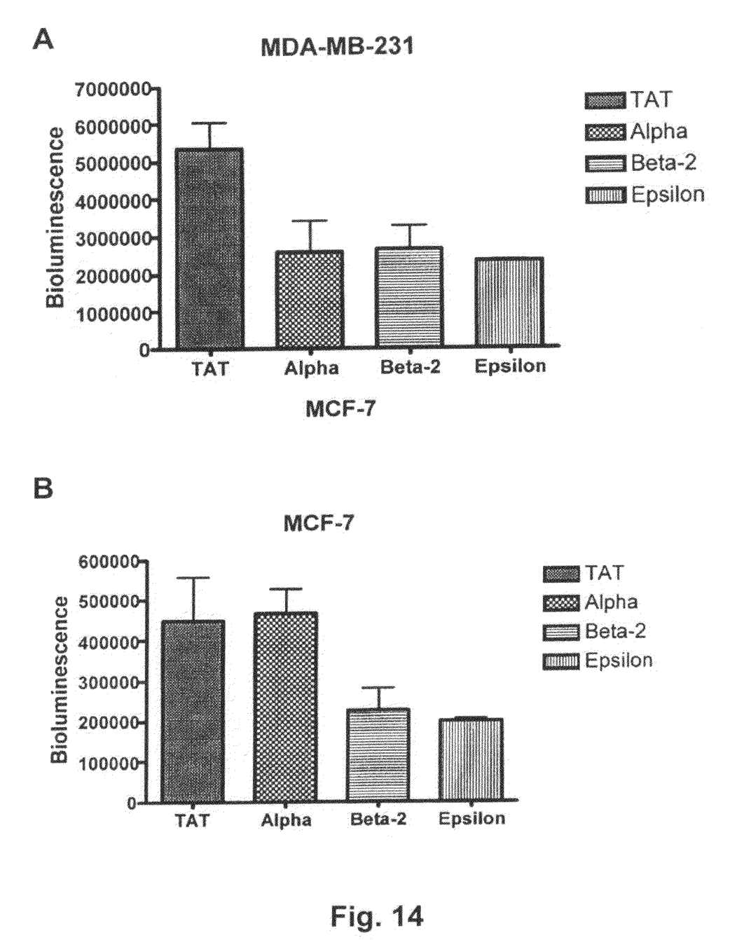

FIGS. 14A and 14B show the results of a related experiment measuring the efficacy of TAT carrier peptide (TAT), αV5-3-TAT peptide (Alpha), βIIV5-3 peptide (Beta-2), and εV1-2 peptide (Epsilon) in inhibiting the invasion of human MDA-MB-231 and MCF-7 breast cancer cells through a HUVEC monolayer. FIGS. 14C and 14D show the results of a similar experiment showing the efficacy of TAT carrier peptide (TAT) or αV5-3-TAT peptide in inhibiting the invasion of 4T1 (FIG. 11C) and MDA-MB-231 (FIG. 11D) cells. The results demonstrated that the αPKC inhibitor reduced the invasion of MDA cells, while the β_{II}PKC inhibitor reduced migration and invasion of both MDA and MCF cells. The εPKC inhibitor also reduce invasion of both the MDA and MCF human breast cancer cells.

Interestingly, although the αPKC inhibitor reduced the metastasis of 4T1 cells in vivo, it did not affect migration of 4T1 cells in vitro. These data suggest the use of caution when interpreting in vitro results, and support the present use of a in vivo animal model for determining the efficacy of PKC inhibitors for reducing metastasis.

### G. Conclusions

The results demonstrated that treatment of animals with an isozyme-specific inhibitor of αPKC reduced metastasis, increased the survival of animals, reduced lung adhesion of tumor cells following injection, reduced MMP2 activity, and reduced CXCR4 receptor expression. Inhibition of αPKC also reversed abnormally elevated level of serum AST/ALT levels (a marker of damage or toxicity of the liver) to the normal range, suggesting that the dose used in this study was not toxic.

The present methods provide PKC inhibitors for inhibiting metastasis in an animal with a tumor, reducing migration of tumor cells from the site of a primary tumor to a remote site; reducing signaling between tumor cells and remote sites in the body; and/or reducing the adhesion of tumor cells to remote sites in the body.

### III. Polypeptides for Use with the Methods

A. αPKC Inhibitors

In some embodiments, the PKC inhibitor is a αPKC inhibitor, such as a αPKC inhibitor peptide having a sequence derived from the V5 domain. An inhibitor of αPKC may be a compound that inactivates αPKC, to form inactive αPKC, prevents αPKC from performing its biological functions, or otherwise antagonizes the activity of αPKC. The antagonist/inhibitor may be a competitive, non-competitive, or uncompetitive inhibitor of β_{II}PKC. In some embodiments, the inhibitor is a selective peptide inhibitor of β_{II}PKC, as opposed to an inhibitor of other PKC isozymes.

The V5 domain of the αPKC isozyme has the amino acid sequence identified herein as SEQ ID NO: 1, taken from amino acid residue 616 et seq. of αPKC. A preferred inhibitor αPKC peptide, corresponding to amino acid residues 620-625 of the αPKC isozyme, is QLVIAN, identified herein as SEQ ID NO: 6. Another exemplary peptide is GKGAEN (SEQ ID NO: 7), corresponding to amino acid residues 620-625. It will be appreciated that peptides homologous to the native sequences and peptides having conservative amino acid substitutions, are within the scope of peptides contemplated. For example, one or two amino acids can be substituted, and exemplary modifications include changing between R and K; between V, L, I, R and D; and/or between G, A, P and N.

The αPKC inhibitor peptide may be derived from the alpha (α)-isozyme of PKC from any species, such as Rattus norvegicus, Homo sapiens (Genbank Accession No. NP_{—}002728) or Bos taurus (Genbank Accession No. NP_{—}776860).

Peptides derived from the V5 domain of αPKC, which are expected to produce an αPKC isozyme-specific peptide inhibitor, include peptides (or their derivatives) such as QiVIAN (SEQ ID NO: 8), QvVIAN (SEQ ID NO: 9), QLVIAa (SEQ ID NO: 10), QLVInN (SEQ ID NO: 11), and QLVIAN (SEQ ID NO: 12).

In particular embodiments, the peptide is a peptide having between about 5 and 15 contiguous residues, more preferably 5-10 contiguous residues, still more preferably 5-8 contiguous residues, from the V5 domain of αPKC.

B. βPKC Inhibitors

In some embodiments, the PKC inhibitor is a βPKC inhibitor, such as a β_{I}PKC or β_{II}PKC inhibitor peptide having a sequence derived from the V5 domain. An inhibitor of PKC may be a compound that inactivates βPKC, to form inactive βPKC, prevents βPKC from performing its biological functions, or otherwise antagonizes the activity of βPKC. The antagonist/inhibitor may be a competitive, non-competitive, or uncompetitive inhibitor of βPKC. In some embodiments, the inhibitor is a selective peptide inhibitor of βPKC, as opposed to an inhibitor of other PKC isozymes.

The V5 domain of the β_{II}PKC isozyme has the amino acid sequence: “PKACGRNAENFDRFFTRHPPVLTPPDQEVIRNIDQSEFEGFSFVNSEFLKPEVKS” (SEQ ID NO: 2). Exemplary peptides include CGRNAE (SEQ ID NO: 13), KACGRNAE (SEQ ID NO: 14) and CGRNAEN (SEQ ID NO: 15) and modified peptide ACGkNAE (SEQ ID NO: 15). Excluded are the peptides ACGRNAE (SEQ ID NO:17) QEVIRN (SEQ ID NO: 18) and SFVNSEFLKPEVKS (SEQ ID NO: 19).

The βPKC inhibitor peptide may be derived from the beta I or II (β_{I} or β_{II})-isozyme of PKC from any species, such as Rattus norvegicus (Genbank Accession No. NP_{—}036845) or Homo sapiens (Genbank Accession No. AAD138520; BAA00912, CAA05725; CAA44393).

In particular embodiments, the peptide is a peptide having between about 5 and 15 contiguous residues, more preferably 5-10 contiguous residues, still more preferably 5-8 contiguous residues, from the V5 region of βPKC.

C. εPKC Inhibitors

In some embodiments, the PKC inhibitor is a εPKC inhibitor, such as a εPKC inhibitor peptide having a sequence derived from the V5 domain. An inhibitor of εPKC may be a compound that inactivates εPKC, to form inactive εPKC, prevents εPKC from performing its biological functions, or otherwise antagonizes the activity of εPKC. The antagonist/inhibitor may be a competitive, non-competitive, or uncompetitive inhibitor of εPKC. In some embodiments, the inhibitor is a selective peptide inhibitor of εPKC, as opposed to an inhibitor of other PKC isozymes.

The polypeptide sequences of murine, rat, and human εPKC are reproduced, below. The present compositions and methods contemplate the use of any one of these polypeptides, chimeric/hybrid polypeptides including sequence from one or more of these polypeptides, and/or fragments, variants, and derivatives, thereof.

εPKC (Mus musculus); gi: 6755084; ACCESSION: NP_{—}035234 XP_{—}994572 XP_{—}994601 XP_{—}994628 (SEQ ID NO: 3):

| | |
|---|---|
| 1 | MVVFNGLLKI KICEAVSLKP TAWSLRHAVG PRPQTFLLDP |
| | YIALNVDDSR IGQTATKQKT |
| 61 | MSPAWHDEFV TDVCNGRKIE LAVFHDAPIG YDDFVANCTI |
| | QFEELLQNGS RHFEDWIDLE |
| 121 | PEGKVYVIID LSGSSGEAPK DNEERVFRER MRPRKRQGAV |
| | RRRVHQVNGH KFMATYLRQP |
| 181 | TYCSHCRDFI WGVIGKQGYQ CQVCTCVVHK RCHELIITKC |
| | AGLKKQETPD EVCSQRFSVN |
| 241 | MPHKFGIHNY KVPTFCDHCG SLLWGLLRQG LQCKVCKMNV |
| | HRRCETNVAP NCGVDARGIA |
| 301 | KVLADLGVTP DKITNSGQRR KKLAAGAESP QPASGNSPSE |
| | DDRSKSAPTS PCDQELKELE |
| 361 | NNIRKALSFD NRGEEHRASS ATDGQLASPG ENGEVRPGQA |
| | KRLGLDEFNF IKVLGKGSFG |
| 421 | KVMLAELKGK DEVYAVKVLK KDVILQDDDV DCTMTEKRIL |
| | ALARXHPYLT QLYCCFQTKD |
| 481 | RLFFVMEYVN GGDLMFQIQR SRXFDEPRSR FYAAEVTSAL |
| | MFLHQHGVIY RDLKLDNILL |
| 541 | DAEGHCKLAD FGMCKEGIMN GVTTTTFCGT PDYIAPEILQ |
| | ELEYGPSVDW WALGVLMYEM |
| 601 | MAGQPPFEAD NEDDLFESIL HDDVLYPVWL SKEAVSILKA |
| | FMTKNPHKRL GCVAAQNGED |
| 661 | AIKQHPFFKE IDWVLLEQKK IKPPFKPRIK TKRDVNNFDQ |
| | DFTREEPILT LVDEAIIKQI |
| 721 | NQEEFKGFSY FGEDLMP |

εPKC (Rattus norvegicus); ACCESSION: NP_{—}058867 XP_{—}343013 (SEQ ID NO: 4):

| | |
|---|---|
| 1 | MVVFNGLLKI KICEAVSLKP TAWSLRHAVG PRPQTFLLDP |
| | YIALNVDDSR IGQTATKQKT |
| 61 | NSPAWHDEFV TDVCNGRKIE LAVFHDAPIG YDDFVANCTI |
| | QFEELLQNGS RHFEDWIDLE |
| 121 | PEGKVYVIID LSGSSGEAPK DNEERVFRER MRPRKRQGAV |
| | RRRVHQVNCH KFMATYLRQP |
| 181 | TYCSHCRDFI WGVIGKQGYQ CQVCTCVVHK RCHELIITKC |
| | AGLKKQETPD EVGSQRFSVN |
| 241 | MPHXFGIHNY KVPTFCDHCG SLLWGLLRQG LQCKVCKMNV |
| | HRRCETNVAP NCGVDARGIA |
| 301 | KVLADLGVTP DKITNSGQRR KKLAAGAESP QPASGNSPSE |
| | DDRSKSAPTS PCDQELKELE |
| 361 | NNIRXALSFD NRGEEHRASS STDGQLASPG ENGEVRQGQA |
| | KRLGLDEFNF IKVLGKGSFG |
| 421 | KVMLAELKGK DEVYAVKVLK KDVILQDDDV DCTMTEKRIL |
| | ALARKHPYLT QLYCCFQTKD |
| 481 | RLFFVMEYVN GGDLMFQIQR SRKFDEPRSG FYAAEVTSAL |
| | MFLHQHGVIY RDLKLDNILL |
| 541 | DAEGHSKLAD FGMCKEGILN GVTTTTFCCT PDYIAPEILQ |
| | ELEYCPSVDW WALCVLMYEM |
| 601 | MAGQPPFEAD NEDDLFESIL HDDVLYPVWL SKEAVSILKA |
| | FMTKNPHKRL CCVAAQNGED |
| 661 | AIKQHPFFKE IDWVLLEQKK MKPPFKPRIK TKRDVNNFDQ |
| | DFTREEPILT LVDEAIVKQI |
| 721 | NQEEFKGFSY PGEDLMP |

εPKC (Homo sapiens); ACCESSION: NP_{—}005391 (SEQ ID NO: 5):

| | |
|---|---|
| 1 | mvvfngllki kiceavslkp tawslrhavg prpqtflldp |
| | yialnvddsr igqtatkqkt |
| 61 | nspawhdefv tdvcngrkie lavfhdapig yddfvancti |
| | qfeellqngs rhfedwidle |
| 121 | pegrvyviid lsgssgeapk dneervfrer mrprkrqgav |
| | rrrvhqvngh kfmatylrqp |
| 181 | tycshcrdfi wgvigkqgyq cqvctcvvhk rcheliitkc |
| | aglkkqetpd qvgsqrfsvn |
| 241 | mphkfgihny kvptfcdhcg sllwgllrqg lqckvckmnv |
| | hrrcetnvap ncgvdargia |
| 301 | kvladlgvtp dkitnsgqrr kkliagaesp qpasgsspse |
| | edrsksapts pcdqeikele |
| 361 | nnirkalsfd nrgeehraas spdgqlmspg engevrqgqa |
| | krlgldefnf ikvlgkgsfg |
| 421 | kvmlaelkgk devyavkvlk kdvilqdddv dctmtekril |
| | alarkhpylt qlyccfqtkd |
| 481 | rlffvmeyvn ggdlmfqiqr srkfdeprsr fyaaevtsal |
| | mflhqhgviy rdlkldnill |
| 541 | daeghcklad fgmckegiln gvttttfcgt pdyiapeilq |
| | eleygpsvdw walgvlmyem |
| 601 | magqppfead neddifesil hddvlypvwl skeavsilka |
| | fmtknphkrl gcvasqtiged |
| 661 | aikqhpffke idwvlleqkk ikppfkprik tkrdvnnfdq |
| | dftreepvlt lvdeaivkqi |
| 721 | nqeefkgfsy fgedlmp |

An exemplary εPKC inhibitor peptide is TAT₄₇₋₅₇-εV1-2, which contains amino acid residues 47-57 of the HIV TAT transactivator protein, which directs entry into cells, and amino acid residues 14-21 of εPKC (i.e., EAVSLKPT; SEQ ID NO: 20). This εPKC inhibitor is described in Chen, L. et al. ((2001) Chem. Biol. 8:1123-9) and in U.S. Publication Nos. US2004-0009919A1, US2005-0209160A1, US2005-0164947A1, US2006-0148700A1, which further describe the characterization of εPKC agonists and antagonists and which are incorporated by reference herein. Other εPKC inhibitor peptides may be used, including but not limited to peptides containing conservative amino acid substitutions and peptides having similarity to εPKC RACK amino acid residues, as described, below.

In εPKC, the sequence HDAPIGYD (SEQ ID NO: 21; εPKC 85-92; Genbank Accession No. NP_{—}058867), named ψεRACK, has 75% homology with a sequence in εRACK consisting of amino acids NNVALGYD (RACK 285-292; SEQ ID NO: 22). A peptide corresponding to the ψεRACK sequence functioned as a εPKC-selective agonist (Dorn, G. W. et al. (1999) Proc. Natl. Acad. Sci. U.S.A. 96:12798-12803), possibly by stabilizing the “open” form of εPKC. Mutating Asp-86 in the ψεRACK sequence of εPKC to an Asn (i.e., D→N) produced an enzyme that translocated more slowly than the wild-type enzyme, presumably due to increased intramolecular interaction between the εRACK and the mutated ψεRACK-binding site in εPKC, which stabilized the “closed form.” Accordingly, the mutated ψεRACK peptide having the amino acid sequence HNAPIGYD (SEQ ID NO: 23), functioned as a εPKC antagonist/inhibitor (Schechtman, D. et al. (2004) J. Biol. Chem. 279:15831-15840; Liron, T. et al. (2007) J. Molecular and Cellular Cardiology 42:835-841). Other mutated ψεRACK are expected to function as εPKC antagonists/inhibitors. In addition, the polypeptide β′-COP has an εPKC binding motif (i.e., NNVALGYD; SEQ ID NO: 24), which is expected to function as an antagonist/inhibitor of εPKC (Dorn et al. (1999) Proc. Natl. Acad. Sci., USA; Schechtman et al (2004) J. Biol. Chem).

In particular embodiments, the peptide is a peptide having between about 5 and 15 contiguous residues, more preferably 5-10 contiguous residues, still more preferably 5-8 contiguous residues, from the V5 region of εPKC.

D. Variant and Modified Polypeptides

The peptide inhibitors described herein also encompass amino acid sequences similar to the amino acid sequences set forth herein that have at least about 50% identity thereto and function to inhibit tumor growth and/or angiogenesis. Preferably, the amino acid sequences of the peptide inhibitors encompassed in the invention have at least about 60% identity, further at least about 70% identity, preferably at least about 75% or 80% identity, more preferably at least about 85% or 90% identity, and further preferably at least about 95% identity, to the amino acid sequences set forth herein. Percent identity may be determined, for example, by comparing sequence information using the advanced BLAST computer program, including version 2.2.9, available from the National Institutes of Health. The BLAST program is based on the alignment method of Karlin and Altschul ((1990) Proc. Natl. Acad. Sci. USA 87:2264-68) and as discussed in Altschul et al. ((1990) J. Mol. Biol. 215:403-10; Karlin and Altschul (1993) Proc. Natl. Acad. Sci. USA 90:5873-77; and Altschul et al. (1997) Nucleic Acids Res. 25:3389-3402).

Conservative amino acid substitutions may be made in the amino acid sequences described herein to obtain derivatives of the peptides that may advantageously be utilized in the present invention. Conservative amino acid substitutions, as known in the art and as referred to herein, involve substituting amino acids in a protein with amino acids having similar side chains in terms of, for example, structure, size and/or chemical properties. For example, the amino acids within each of the following groups may be interchanged with other amino acids in the same group: amino acids having aliphatic side chains, including glycine, alanine, valine, leucine and isoleucine; amino acids having non-aromatic, hydroxyl-containing side chains, such as serine and threonine; amino acids having acidic side chains, such as aspartic acid and glutamic acid; amino acids having amide side chains, including glutamine and asparagine; basic amino acids, including lysine, arginine and histidine; amino acids having aromatic ring side chains, including phenylalanine, tyrosine and tryptophan; and amino acids having sulfur-containing side chains, including cysteine and methionine. Additionally, amino acids having acidic side chains, such as aspartic acid and glutamic acid, are considered interchangeable herein with amino acids having amide side chains, such as asparagine and glutamine. The PKC peptide inhibitors may also include natural amino acids, such as the L-amino acids or non-natural amino acids, such as D-amino acids.

A wide variety of modifications to the amide bonds which link amino acids may be made and are known in the art. Such modifications are discussed in general reviews, including in Freidinger, R. M. (2003) J. Med. Chem. 46:5553, and Ripka, A. S. and Rich, D. H. (1998) Curr. Opin. Chem. Biol. 2:441. These modifications are designed to improve the properties of the peptide by increasing the potency of the peptide or by increasing the half-life of the peptide.

The inhibitors may be pegylated, which is a common modification to reduce systemic clearance with minimal loss of biological activity. Polyethylene glycol polymers (PEG) may be linked to various functional groups of PKC peptide inhibitor polypeptides/peptides using methods known in the art (see, e.g., Roberts et al. (2002), Advanced Drug Delivery Reviews 54:459-76 and Sakane et al. (1997) Pharm. Res. 14:1085-91). PEG may be linked to, e.g., amino groups, carboxyl groups, modified or natural N-termini, amine groups, and thiol groups. In some embodiments, one or more surface amino acid residues are modified with PEG molecules. PEG molecules may be of various sizes (e.g., ranging from about 2 to 40 kDa). PEG molecules linked to PKC peptide inhibitor may have a molecular weight about any of 2,000, 10,000, 15,000, 20,000, 25,000, 30,000, 35,000, 40,000 Da. PEG molecule may be a single or branched chain. To link PEG to PKC peptide inhibitor, a derivative of PEG having a functional group at one or both termini may be used. The functional group is chosen based on the type of available reactive group on the polypeptide. Methods of linking derivatives to polypeptides are known in the art.

In some embodiments, the peptide inhibitor is modified with to achieve an increase in cellular uptake of the peptide inhibitor. Such a modification may be, for example, attachment to a carrier peptide, such as a Drosophila melanogaster Antennapedia homeodomain-derived sequence (unmodified sequence may be found in Genbank Accession No. AAD19795) which is set forth in SEQ ID NO: 29 (RQIKIWFQNRRMKWKK), the attachment being achieved, for example, by cross-linking via an N-terminal Cys-Cys bond as discussed in Theodore, L., et al. J. Neurosci. 15:7158-7167 (1995); Johnson, J. A., et al. Circ. Res 79:1086 (1996). The terminal cysteine residues may be part of the naturally-occurring or modified amino acid sequences or may be added to an amino sequence to facilitate attachment. The carrier peptide sequence may also be sought from Drosophila hydei and Drosophila virilis. Alternatively, the peptide inhibitor may be modified by a Transactivating Regulatory Protein (Tat)-derived transport polypeptide (such as from amino acids 47-57 of Tat shown in SEQ ID NO: 25; YGRKKRRQRRR) from the Human Immunodeficiency Virus, Type 1, as described in Vives, et al., J. Biol. Chem., 272:16010-16017 (1997), U.S. Pat. No. 5,804,604; and as seen in Genbank Accession No. AAT48070, or with polyarginine as described in Mitchell, et al. J. Peptide Res. 56:318-325 (2000) and Rothbard, et al., Nature Med. 6:1253-1257 (2000). The peptide inhibitor may be modified by other methods known to the skilled artisan in order to increase the cellular uptake of the inhibitors.

The inhibitor peptide may be capable of preventing activation of a PKC isozyme, which are activated in vivo by binding to a cognate polypeptide such as a receptor protein (RACK). Regions of homology between the PKC signaling peptide and its RACK are termed “pseudo-RACK” sequences (i-RACK; Ron, D. et al. (1994) Proc. Natl. Acad. Sci. USA 91:839-843; Ron, D. and Mochly-Rosen, D. (1995) Proc. Natl. Acad. Sci. U.S.A. 92:492-496; Dorn, G. W. et al. (1999) Proc. Natl. Acad. Sci. U.S.A. 96:12798-12803; and Souroujon, M. C. and Mochly-Rosen, D. (1998) Nature Biotech. 16:919-924) and typically have a sequence similar to the PKC-binding region of the corresponding RACK. ψRACK sequence corresponding to α, β, and εPKC, or variants thereof, are expected to function as inhibitors of the cognate PKC.

Peptide inhibitors of PKC may be obtained by methods known to the skilled artisan. For example, The peptide inhibitor may be chemically synthesized using various solid phase synthetic technologies known to the art and as described, for example, in Williams, Paul Lloyd, et al. Chemical Approaches to the Synthesis of Peptides and Proteins, CRC Press, Boca Raton, Fla., (1997).

Alternatively, PKC peptide inhibitors may be produced by recombinant technology methods as known in the art and as described, for example, in Sambrook et al., Molecular Cloning: A Laboratory Manual, Cold Springs Harbor laboratory, 2^{nd} ed., Cold Springs Harbor, N.Y. (1989), Martin, Robin, Protein Synthesis: Methods and Protocols, Humana Press, Totowa, N.J. (1998) and Current Protocols in Molecular Biology (Ausubel et al., eds.), John Wiley & Sons, which is regularly and periodically updated. An expression vector may be used to produce the desired peptide inhibitor in an appropriate host cell and the product may then be isolated by known methods. The expression vector may include, for example, the nucleotide sequence encoding the desired peptide wherein the nucleotide sequence is operably linked to a promoter sequence.

While the present treatment method has largely been described in terms of polypeptides/peptide inhibitors, the method includes administering to an animal in need of such treatment a polynucleotide encoding any of the polypeptide/peptide inhibitors described herein. Polynucleotide encoding peptide inhibitors include gene therapy vectors based on, e.g., adenovirus, adeno-associated virus, retroviruses (including lentiviruses), pox virus, herpesvirus, single-stranded RNA viruses (e.g., alphavirus, flavivirus, and poliovirus), etc. Polynucleotide encoding polypeptides/peptide inhibitors further include naked DNA or plasmids operably linked to a suitable promoter sequence and suitable of directing the expression of any of the polypeptides/peptides described, herein. Polypeptides may be encoded by an expression vector, which may include, for example, the nucleotide sequence encoding the desired peptide wherein the nucleotide sequence is operably linked to a promoter sequence.

As defined herein, a nucleotide sequence is “operably linked” to another nucleotide sequence when it is placed in a functional relationship with another nucleotide sequence. For example, if a coding sequence is operably linked to a promoter sequence, this generally means that the promoter may promote transcription of the coding sequence. Operably linked means that the DNA sequences being linked are typically contiguous and, where necessary to join two protein coding regions, contiguous and in reading frame. However, since enhancers may function when separated from the promoter by several kilobases and intronic sequences may be of variable length, some nucleotide sequences may be operably linked but not contiguous. Additionally, as defined herein, a nucleotide sequence is intended to refer to a natural or synthetic linear and sequential array of nucleotides and/or nucleosides, and derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a polypeptide.

Other suitable PKC inhibitors include organic or inorganic compounds, such as peptidomimetic small-molecules.

### IV. Administration and Dosing of PKC Inhibitors

An osmotic pump was used to deliver the PKC inhibitors to experimental animals (see above and the Examples). The osmotic pump allowed a continuous and consistent dosage of PKC inhibitors to be delivered to animals with minimal handling. While an osmotic pump can be used for delivering PKC inhibitors to human or other mammalian patients, other methods of delivery are contemplated.

PKC inhibitors are preferably administered in various conventional forms. For example, the inhibitors may be administered in tablet form for sublingual administration, in a solution or emulsion. The inhibitors may also be mixed with a pharmaceutically-acceptable carrier or vehicle. In this manner, the PKC inhibitors are used in the manufacture of a medicament for reducing hypertension-induced stroke and encephalopathy.

The vehicle may be a liquid, suitable, for example, for parenteral administration, including water, saline or other aqueous solution, or may be an oil or an aerosol. The vehicle may be selected for intravenous or intraarterial administration, and may include a sterile aqueous or non-aqueous solution that may include preservatives, bacteriostats, buffers and antioxidants known to the art. In the aerosol form, the inhibitor may be used as a powder, with properties including particle size, morphology and surface energy known to the art for optimal dispersability. In tablet form, a solid vehicle may include, for example, lactose, starch, carboxymethyl cellulose, dextrin, calcium phosphate, calcium carbonate, synthetic or natural calcium allocate, magnesium oxide, dry aluminum hydroxide, magnesium stearate, sodium bicarbonate, dry yeast or a combination thereof. The tablet preferably includes one or more agents which aid in oral dissolution. The inhibitors may also be administered in forms in which other similar drugs known in the art are administered, including patches, a bolus, time release formulations, and the like.

The inhibitors described herein may be administered for prolonged periods of time without causing desensitization of the patient to the inhibitor. That is, the inhibitors can be administered multiple times, or after a prolonged period of time including one, two or three or more days; one two, or three or more weeks or several months to a patient and will continue to cause an increase in the flow of blood in the respective blood vessel.

The inhibitors may be administered to a patient by a variety of routes. For example, the inhibitors may be administered parenterally, including intraperitoneally; intravenously; intraarterially; subcutaneously, or intramuscularly. The inhibitors may also be administered via a mucosal surface, including rectally, and intravaginally; intranasally; by inhalation, either orally or intranasally; orally, including sublingually; intraocularly and transdermally. Combinations of these routes of administration are also envisioned.

Suitable carriers, diluents and excipients are well known in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament). Some formulations may include carriers such as liposomes. Liposomal preparations include, but are not limited to, cytofectins, multilamellar vesicles and unilamellar vesicles. Excipients and formulations for parenteral and nonparenteral drug delivery are set forth in Remington, The Science and Practice of Pharmacy (2000).

The skilled artisan will be able to determine the optimum dosage. Generally, the amount of inhibitor utilized may be, for example, about 0.0005 mg/kg body weight to about 50 mg/kg body weight, but is preferably about 0.05 mg/kg to about 0.5 mg/kg. The exemplary concentration of the inhibitors used herein are from 3 mM to 30 mM but concentrations from below about 0.01 mM to above about 100 mM (or to saturation) are expected to provide acceptable results.

### V. Compositions and Kits Comprising PKC Inhibitors

The methods may be practiced using peptide and/or peptimimetic inhibitors of PKC, some of which are identified herein. These compositions may be provided as a formulation in combination with a suitable pharmaceutical carrier, which encompasses liquid formulations, tablets, capsules, films, etc. The PKC inhibitors may also be supplied in lyophilized form. The compositions are suitable sterilized and sealed for protection.

Such compositions may be a component of a kit of parts (i.e., kit). In addition to a PKC inhibitor composition, such kits may include administration and dosing instructions, instructions for identifying patients in need of treatment, and instructions for monitoring a patients' response to PKC inhibitor therapy. Where the PKC inhibitor is administered via a pump (as in the animal studies described, herein), the kit may comprise a pump suitable for delivering PKC inhibitors. The kit may also contain a syringe to administer a formulation comprising a PKC inhibitor by a peripheral route.

### VI. Examples

The following examples are illustrative in nature and are in no way intended to be limiting.

Methods

Wound healing assay: Human or mouse breast cancer cell lines were grown in 6-well plates until about 90% confluent. Cells were then grown in serum free media overnight, before media was changed to media +1% FBS and peptide PKC inhibitors added (1 uM), at the same time a pipette tip was drawn across the cell layer to create a scrape ‘wound’. PKC inhibitors were re-applied every 2 h for a total of 8 h, before cells were left for a further 12 h and then the scrape was examined under phase contrast microscopy.

Cell Migration/invasion assay: Primary human endothelial cells (HUVEC's) were plated on top of a matrigel plug in trans-well plates. Once the cells had reached confluence (as determined by examination of a control well), 1×10⁶ human breast cancer cells (MCF-7 or MDA-MB-231) expressing luciferase were added above the HUVEC cell layer. Where applicable, peptides were added to a final concentration of 10 μM and re-applied every 2 hours for 10 hours followed by further incubation. After 24-48 hours-incubation the HUVEC cell layer (and all other cells above the matrigel) were removed by scraping of the cell layer into the media and aspiration (repeated 3 times). The relative numbers of cancer cells that had crossed the HUVEC cell layer and entered the matrigel was then determined by bioluminescence detection of luciferase action following addition of luciferin substrate.

### Example 1

### In Vivo Administration of αPKC Peptide Inhibitor for Inhibition of Metastases

The PKC peptides and TAT₄₇₋₅₇ were synthesized and conjugated via a Cys S-S bond as described previously (Chen, et al. (2001) Proc. Natl. Acad. Sci. USA 25:11114-19 and Inagaki, et al. (2003) Circulation 11:2304-07).

Balb/c female mice (6 weeks old) were injected semi-orthotopically with 4T1 murine mammary cancer cells tagged with luciferase (100,0000 cells/100 μL). One week later, an osmotic minipump was implanted subcutaneously in each animal, for delivery of saline (control), TAT peptide (control, YGRKKRRQRRR, SEQ ID NO: 25), the peptide inhibitor αV5-3 (QLVIAN, SEQ ID NO: 6) attached via an N-terminal disulfide bond to TAT peptide (YGRKKRRQRRR-CC-QLVIAN, SEQ ID NO: 26).

In other experiments, the peptide inhibitor β_{II}V5-3 (QEVIAN, SEQ ID NO: 13) or εV1-2 (EAVSLKPT, SEQ ID NO: 20) is attached via an N-terminal disulfide bond to the TAT peptide (SEQ ID NOs: 27 and 28, respectively). The TAT control, αV5-3-TAT, βIIV5-3-TAT, or εV1-2-TAT conjugate peptides are administered at about 35 mg/kg/day for two and four weeks.

After two weeks of treatment with αV5-3-TAT, some animals were selected form imaging (IVIS® 29, Xenogen Corporation, Alameda, Calif.) and analysis, with the remaining animals imaged after four weeks of treatment.

FIG. 3 shows images of a representative mouse from each group of mice treated for four weeks with saline, TAT peptide, and αV5-3-TAT conjugate peptide.

FIG. 4 is a bar graph showing the extent of lung metastasis, expressed as relative light units (based on imaging as exemplified in FIG. 3), in tumor-bearing mice treated for four weeks with saline, the αV5-3-TAT conjugate peptide (αPKC), or the β_{II}V5-3-TAT conjugate peptide (βPKC). The extent of lung metastasis, quantified approximately by the relative light units, for mice treated with saline, the αPKC inhibitor, and the β_{II}V5-3 PKC inhibitor peptide is shown. The extent of lung metastases is reduced in the animals treated with the αPKC inhibitor. Treatment with βIIV5-3 had a less pronounced effect.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A method for inhibiting metastasis comprising administering to a patient with a tumor an effective amount of a protein kinase C (PKC) inhibitor.

2. The method of claim 1, wherein the PKC inhibitor is an αPKC inhibitor.

3. The method of claim 2, wherein the αPKC inhibitor comprises an amino acid sequence from the αPKC V5 domain.

4. The method of claim 3, wherein the αPKC inhibitor comprises the αV5-3 peptide (SEQ ID NO: 6).

5. The method of claim 4, wherein the αV5-3 peptide is conjugated to a peptide for increase cell permeability.

6. The method of claim 1, wherein the PKC inhibitor is a β_{II}PKC inhibitor.

7. The method of claim 6, wherein the β_{II}PKC inhibitor comprises an amino acid sequence from the β_{II}PKC V5 domain.

8. The method of claim 7, wherein the αPKC inhibitor comprises the β_{II}v5-3 peptide (SEQ ID NO: 13).

9. The method of claim 8, wherein the β_{II}v5-3 peptide is conjugated to a peptide for increase cell permeability.

10. The method of claim 1, wherein the PKC inhibitor is an εPKC inhibitor.

11. The method of claim 10, wherein the εPKC inhibitor comprises an amino acid sequence from amino acid residues 14-21 of εPKC (SEQ ID NO: 20).

12. The method of claim 11, wherein the ε1-2 peptide is conjugated to a peptide for increase cell permeability.

13. The method of claim 1, wherein the tumor is a breast cancer tumor.

14. The method of claim 1, wherein the tumor is a mammary cancer tumor.


SEQUENCE LISTING (29 sequences)

SEQ ID NO: 1 - protein, 57 aa, Artificial Sequence
  V5 domain of human alpha PKC
PKVCGKGAENFDKFFTRGQPVLTPPDQLVIANIDQSDFEGFSYVNPQFVHPILQSAV

SEQ ID NO: 2 - protein, 55 aa, Artificial Sequence
  V5 domain of human beta-II PKC
PKACGRNAENFDRFFTRHPPVLTPPDQEVIRNIDQSEFEGFSFVNSEFLKPEVKS

SEQ ID NO: 3 - protein, 737 aa, Mus musculus
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGKVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDEVGSQRFSVNMPHKFGIHNYKVPTFCDHCGSLLWGLLRQGLQCKVCKMNVHRRCETNVAPNCGVDARGIAKVLADLGVTPDKITNSGQRRKKLAAGAESPQPASGNSPSEDDRSKSAPTSPCDQELKELENNIRKALSFDNRGEEHRASSATDGQLASPGENGEVRPGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSRFYAAEVTSALMFLHQHGVIYRDLKLDNILLDAEGHCKLADFGMCKEGIMNGVTTTTFCGTPDYIAPEILQELEYGPSVDWWALGVLMYEMMAGQPPFEADNEDDLFESILHDDVLYPVWLSKEAVSILKAFMTKNPHKRLGCVAAQNGEDAIKQHPFFKEIDWVLLEQKKIKPPFKPRIKTKRDVNNFDQDFTREEPILTLVDEAIIKQINQEEFKGFSYFGEDLMP

SEQ ID NO: 4 - protein, 737 aa, Rattus norvegicus
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGKVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDEVGSQRFSVNMPHKFGIHNYKVPTFCDHCGSLLWGLLRQGLQCKVCKMNVHRRCETNVAPNCGVDARGIAKVLADLGVTPDKITNSGQRRKKLAAGAESPQPASGNSPSEDDRSKSAPTSPCDQELKELENNIRKALSFDNRGEEHRASSSTDGQLASPGENGEVRQGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSGFYAAEVTSALMFLHQHGVIYRDLKLDNILLDAEGHSKLADFGMCKEGILNGVTTTTFCGTPDYIAPEILQELEYGPSVDWWALGVLMYEMMAGQPPFEADNEDDLFESILHDDVLYPVWLSKEAVSILKAFMTKNPHKRLGCVAAQNGEDAIKQHPFFKEIDWVLLEQKKMKPPFKPRIKTKRDVNNFDQDFTREEPILTLVDEAIVKQINQEEFKGFSYFGEDLMP

SEQ ID NO: 5 - protein, 737 aa, Homo sapiens
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGRVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDQVGSQRFSVNMPHKFGIHNYKVPTFCDHCGSLLWGLLRQGLQCKVCKMNVHRRCETNVAPNCGVDARGIAKVLADLGVTPDKITNSGQRRKKLIAGAESPQPASGSSPSEEDRSKSAPTSPCDQEIKELENNIRKALSFDNRGEEHRAASSPDGQLMSPGENGEVRQGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSRFYAAEVTSALMFLHQHGVIYRDLKLDNILLDAEGHCKLADFGMCKEGILNGVTTTTFCGTPDYIAPEILQELEYGPSVDWWALGVLMYEMMAGQPPFEADNEDDLFESILHDDVLYPVWLSKEAVSILKAFMTKNPHKRLGCVASQNGEDAIKQHPFFKEIDWVLLEQKKIKPPFKPRIKTKRDVNNFDQDFTREEPVLTLVDEAIVKQINQEEFKGFSYFGEDLMP

SEQ ID NO: 6 - protein, 6 aa, Artificial Sequence
  Synthetic peptide
QLVIAN

SEQ ID NO: 7 - protein, 6 aa, Artificial Sequence
  Synthetic peptide
GKGAEN

SEQ ID NO: 8 - protein, 6 aa, Artificial Sequence
  Synthetic peptide
QIVIAN

SEQ ID NO: 9 - protein, 6 aa, Artificial Sequence
  Synthetic peptide
QVVIAN

SEQ ID NO: 10 - protein, 6 aa, Artificial Sequence
  Synthetic peptide
QLVIAA

SEQ ID NO: 11 - protein, 6 aa, Artificial Sequence
  Synthetic peptide
QLVINN

SEQ ID NO: 12 - protein, 6 aa, Artificial Sequence
  Synthetic peptide
QLVIAN

SEQ ID NO: 13 - protein, 6 aa, Artificial Sequence
  Synthetic peptide
CGRNAE

SEQ ID NO: 14 - protein, 8 aa, Artificial Sequence
  Synthetic peptide
KACGRNAE

SEQ ID NO: 15 - protein, 7 aa, Artificial Sequence
  Synthetic peptide
CGRNAEN

SEQ ID NO: 16 - protein, 7 aa, Artificial Sequence
  Synthetic peptide
ACGKNAE

SEQ ID NO: 17 - protein, 7 aa, Artificial Sequence
  Synthetic peptide
ACGRNAE

SEQ ID NO: 18 - protein, 6 aa, Artificial Sequence
  Synthetic peptide
QEVIRN

SEQ ID NO: 19 - protein, 14 aa, Artificial Sequence
  Synthetic peptide
SFVNSEFLKPEVKS

SEQ ID NO: 20 - protein, 8 aa, Artificial Sequence
  Synthetic peptide
EAVSLKPT

SEQ ID NO: 21 - protein, 8 aa, Artificial Sequence
  Synthetic peptide
HDAPIGYD

SEQ ID NO: 22 - protein, 8 aa, Artificial Sequence
  Synthetic peptide
NNVALGYD

SEQ ID NO: 23 - protein, 8 aa, Artificial Sequence
  Synthetic peptide
HNAPIGYD

SEQ ID NO: 24 - protein, 8 aa, Artificial Sequence
  Synthetic peptide
NNVALGYD

SEQ ID NO: 25 - protein, 11 aa, Artificial Sequence
  TAT carrier peptide from HIV Type 1
YGRKKRRQRRR

SEQ ID NO: 26 - protein, 19 aa, Artificial Sequence
  Synthetic peptide
YGRKKRRQRRRCCQLVIAN

SEQ ID NO: 27 - protein, 19 aa, Artificial Sequence
  Synthetic Polypeptide
YGRKKRRQRRRCCCGRNAE

SEQ ID NO: 28 - protein, 21 aa, Artificial Sequence
  Synthetic Polypeptide
YGRKKRRQRRRCCEAVSLKPT

SEQ ID NO: 29 - protein, 16 aa, Artificial Sequence
  Drosophila Antennapedia homeodomain-derived carrier
RQIKIWFQNRRMKWKK